(19) United States
(12) Patent Application Publication
(10) Pub. No.: US 2025/0344936 A1
(43) Pub. Date: Nov. 13, 2025

(54) PROCESSOR DEVICE

(71) Applicant: FUJIFILM Corporation, Tokyo (JP)

(72) Inventor: Takuto YANAGISAWA, Kanagawa (JP)

(73) Assignee: FUJIFILM Corporation, Tokyo (JP)

(21) Appl. No.: 19/199,321

(22) Filed: May 5, 2025

(30) Foreign Application Priority Data
May 8, 2024 (JP) 2024-075730

(51) Int. Cl. A61B 1/00; A61B 1/04; A61B 34/20; A61B 90/00

(52) U.S. Cl. CPC A61B 1/00057; A61B 1/000096; A61B 1/00045; A61B 1/04; A61B 34/20; A61B 2034/2065; A61B 2090/067

(57) ABSTRACT

There is provided a processor device that performs a type determination method for an endoscope by a processor using an endoscope color adjustment jig, the endoscope color adjustment jig including an insertion portion that includes at least one insertion hole into which a distal end portion of the endoscope is inserted, a lid portion that is disposed to face the insertion portion in an insertion direction of the distal end portion, and an outer peripheral portion that is in contact with the insertion portion and the lid portion to form an internal space, the lid portion including a chart that includes a mark on an inner surface facing the internal space, in which the processor is configured to, based on a shape of the mark imaged by the endoscope inserted from the insertion portion, determine an oblique angle of the endoscope.

### CROSS-REFERENCE TO RELATED APPLICATIONS

This application claims priority under 35 U.S.C § 119 (a) to Japanese Patent Application No. 2024-075730 filed on 8 May 2024. The above application is hereby expressly incorporated by reference, in its entirety, into the present application.

### BACKGROUND OF THE INVENTION

### 1. Field of the Invention

The disclosure relates to a processor device.

### 2. Description of the Related Art

In the medical field, endoscopes are used for internal observation, endoscopic surgery, and the like. For the endoscope, including an imaging element for imaging an observation target, various electronic components for generating an image through imaging, and the like are used. These components and the like may have individual differences, degradation over time, or the like, which may result in not obtaining an appropriate image in their original state. Additionally, a light source used for imaging the observation target may also undergo individual differences, degradation over time, or the like. In order to resolve these issues and obtain an appropriate image, calibration, such as white balancing, is performed on the endoscope.

For white balance adjustment, for example, in rigid scopes used in endoscopic surgery and the like, white balance adjustment may be performed by imaging a white sterilized gauze. In addition, a method is known in which white balance adjustment is performed by providing a white cap on a distal end portion of an endoscope (JP2020-032201A (corresponding to US2014/267656A1) and JP2010-200880A). Further, it is known that white balance adjustment is performed using a cap-shaped white balance adjustment tool (JP1993-076483A (JP-H05-076483A)).

### SUMMARY OF THE INVENTION

Endoscopes used in the medical field have various oblique angles and outer diameters depending on their use, purpose, and the like. Therefore, in a case in which image signal (image color) correction (hereinafter, referred to as image signal correction), such as white balance adjustment and device variation correction, is performed using an image captured by an endoscope (hereinafter, referred to as an endoscope image), it is necessary to perform the image signal correction according to endoscopes with the various oblique angles and outer diameters. In other words, by more appropriately determining the type of endoscope, image signal correction can be performed with greater accuracy.

An object of the disclosure is to provide a processor device that more appropriately determines a type of endoscope.

According to an exemplary embodiment of the invention, there is provided a processor device that images a chart including a mark provided in an endoscope color adjustment jig, and determines an oblique angle of an endoscope by a processor, the processor being configured to: based on a shape of the mark imaged by the endoscope inserted into the endoscope color adjustment jig, determine the oblique angle of the endoscope.

It is preferable that the endoscope color adjustment jig includes an insertion portion that includes at least one insertion hole into which a distal end portion of the endoscope is inserted, a lid portion that is disposed to face the insertion portion in an insertion direction of the distal end portion, and an outer peripheral portion that is in contact with the insertion portion and the lid portion to form an internal space with the insertion portion and the lid portion, the lid portion includes the chart on an inner surface facing the internal space, and the processor is configured to, based on a shape of an inner side surface of the outer peripheral portion imaged by the endoscope, determine the oblique angle of the endoscope.

It is preferable that the processor is configured to display, on a display, an imaging guide for adjusting a distance between the endoscope and the mark in a case in which the mark is imaged by the endoscope inserted from the insertion portion. Here, it is more preferable that the processor is configured to issue a notification of an imaging state based on a comparison result between the shape of the mark imaged by the endoscope and the imaging guide.

It is preferable that the processor is configured to, based on the shape of the mark in at least one frame of a video obtained by imaging the mark with the endoscope, determine the oblique angle of the endoscope.

It is preferable that the processor is configured to, based on a still image obtained by imaging the mark with the endoscope in response to a still image acquisition instruction, determine the oblique angle of the endoscope.

It is preferable that the processor is configured to, based on a comparison result between a feature amount of the shape of the mark imaged by the endoscope and a feature amount for comparison, determine the oblique angle of the endoscope.

It is preferable that the processor is configured to use a learning model trained on the shape of the mark through machine learning to, based on the shape of the mark imaged by the endoscope, determine the oblique angle of the endoscope.

It is preferable that the processor is configured to output a result of the determination of the oblique angle of the endoscope to an outside.

It is preferable that the processor is configured to decide on a correction parameter for correcting a captured image signal obtained by imaging the mark with the endoscope, based on a result of the determination of the oblique angle of the endoscope.

It is preferable that the processor is configured to set a position of the imaging guide based on a result of the determination of the oblique angle of the endoscope. Here, it is more preferable that the processor is configured to issue a notification of an imaging state based on a comparison result between the shape of the mark imaged by the endoscope and the imaging guide.

It is preferable that the endoscope color adjustment jig is disposed such that the chart is inclined with respect to a perpendicular plane perpendicular to an insertion direction of a distal end portion of the endoscope.

It is preferable that the endoscope color adjustment jig is disposed such that the chart is inclined with respect to a perpendicular plane perpendicular to the insertion direction of the distal end portion of the endoscope, and an inclination angle β of the endoscope color adjustment jig is set in advance such that, in a case in which the distal end portion of the endoscope inserted into the insertion portion is rotated in a circumferential direction, a difference appears in the shape of the mark imaged by endoscopes with different oblique angles α.

It is preferable that the endoscope color adjustment jig is disposed such that the chart is inclined with respect to a perpendicular plane perpendicular to the insertion direction of the distal end portion of the endoscope, and a position of the mark on the chart is set in advance such that, in a case in which the distal end portion of the endoscope inserted into the insertion portion is rotated in a circumferential direction, a difference appears in the shape of the mark imaged by endoscopes with different oblique angles α.

It is preferable that the processor is configured to issue a notification such that a circumferential position of the distal end portion of the endoscope is positioned and the distal end portion is inserted into the insertion portion, before determining the oblique angle.

It is preferable that the chart of the endoscope color adjustment jig includes a plurality of the marks.

In addition, according to another exemplary embodiment of the invention, there is provided a processor device that performs imaging using an endoscope color adjustment jig, and determines an outer diameter type of an endoscope by a processor, the endoscope color adjustment jig including insertion portions that include at least two types of insertion holes with different diameters, into which distal end portions of endoscopes with different outer diameters are inserted, a lid portion that is disposed to face the insertion portions in an insertion direction of the distal end portion, and an outer peripheral portion that is in contact with the insertion portions and the lid portion to form an internal space with the insertion portions and the lid portion, the lid portion including a chart that includes at least two marks facing the respective insertion portions on an inner surface facing the internal space, the marks being located at positions set in advance on the chart such that differences appear in a shape of the mark and a shape of an inner side surface of the outer peripheral portion, which are imaged by endoscopes of different outer diameter types that have been inserted into the insertion portions, the processor being configured to: based on the shape of the mark and the shape of the inner side surface of the outer peripheral portion, which are imaged by the endoscope inserted from the insertion portion, determine the outer diameter type of the endoscope.

It is preferable that the processor is configured to display, on a display, an imaging guide for adjusting a distance between the endoscope and the mark in a case in which the mark is imaged by the endoscope inserted from the insertion portion.

It is preferable that the processor is configured to issue a notification of an imaging state based on a comparison result between the shape of the mark imaged by the endoscope and the imaging guide.

It is preferable that the processor is configured to, based on the shape of the mark and the shape of the inner side surface of the outer peripheral portion in at least one frame of a video obtained by imaging the mark with the endoscope, determine the outer diameter type of the endoscope.

It is preferable that the processor is configured to, based on a designated image obtained by imaging the mark and the inner side surface of the outer peripheral portion with the endoscope in response to a still image acquisition instruction, determine the outer diameter type of the endoscope.

It is preferable that the processor is configured to, based on a comparison result between feature amounts of the shape of the mark and the shape of the inner side surface of the outer peripheral portion, which are imaged by the endoscope, and feature amounts for comparison, determine the outer diameter type of the endoscope.

It is preferable that the processor is configured to use a learning model trained on the shape of the mark and the shape of the inner side surface of the outer peripheral portion through machine learning to, based on the shape of the mark and the shape of the inner side surface of the outer peripheral portion, which are imaged by the endoscope, determine the outer diameter type of the endoscope.

It is preferable that the processor is configured to output a result of the determination of the outer diameter type of the endoscope to an outside.

It is preferable that the processor is configured to decide on a correction parameter for correcting a captured image signal obtained by imaging the mark with the endoscope, based on a result of the determination of the oblique angle of the endoscope.

It is preferable that the processor is configured to set a position of the imaging guide based on a result of the determination of the oblique angle of the endoscope. Here, it is more preferable that the processor is configured to issue a notification of an imaging state based on a comparison result between the shape of the mark imaged by the endoscope and the imaging guide.

It is preferable that the positions of the marks on the chart are set in advance such that, in a case in which the distal end portion of the endoscope inserted into the insertion portion is rotated in a circumferential direction, a difference appears in the shape of the mark imaged by endoscopes with different oblique angles α.

It is preferable that the endoscope color adjustment jig is disposed such that the chart is inclined with respect to a perpendicular plane perpendicular to the insertion direction of the endoscope.

It is preferable that the chart of the endoscope color adjustment jig includes a plurality of the marks corresponding to the respective insertion portions.

According to the exemplary embodiments of the invention, it is possible to determine, based on an image captured by an endoscope, a type of the endoscope.

### BRIEF DESCRIPTION OF THE DRAWINGS

FIG. 1 is a block diagram of an endoscope system.

FIGS. 2A and 2B show a distal end portion of an endoscope, in which FIG. 2A is an explanatory view illustrating an oblique-view scope and FIG. 2B is an explanatory view illustrating a direct-view scope.

FIG. 3 is a perspective view of an endoscope white balance adjustment jig showing a lid portion.

FIG. 4 is a perspective view of the endoscope white balance adjustment jig showing insertion holes.

FIG. 5 is an explanatory view of a case in which the distal end portion of the endoscope is inserted into an insertion hole with a larger inner diameter.

FIG. 6 is an explanatory view of a case in which the distal end portion of the endoscope is inserted into an insertion hole with a smaller inner diameter.

FIG. 7 is a cross-sectional view taken along line I-I of FIG. 6.

FIG. 8 is a cross-sectional view taken along line II-II of FIG. 6.

FIG. 9 is an explanatory view illustrating separation of the lid portion.

FIGS. 10A and 10B show the lid portion, in which FIG. 10A is an explanatory view illustrating an outer surface of the lid portion and FIG. 10B is an explanatory view illustrating an inner surface of the lid portion.

FIGS. 11A to 11C show images captured by the distal end portion of the endoscope, in which FIG. 11A is an image in a case in which a distance between a chart and the distal end portion is appropriate, FIG. 11B is an image in a case in which a position of the distal end portion is too far from the chart, and FIG. 11C is an image in a case in which the position of the distal end portion is too close to the chart.

FIGS. 12A1 to 12C2 are diagrams illustrating an oblique angle determination based on captured images of the endoscopes.

FIG. 13 is a flowchart illustrating the oblique angle determination based on the captured images of the endoscopes.

FIG. 14 is a flowchart illustrating the oblique angle determination based on the captured images of the endoscopes and creation of a white balance adjustment parameter.

FIGS. 15A to 15C show images captured using an adjustment jig with an inclination angle β=22.5 degrees, in which FIG. 15A is an image captured by a direct-view scope, FIG. 15B is an image captured by an oblique-view scope with an oblique angle α=30 degrees, and FIG. 15C is an image captured by an oblique-view scope with an oblique angle α=45 degrees.

FIGS. 16A1 to 16C2 are diagrams illustrating an oblique angle determination based on captured images of the endoscopes.

FIGS. 17A1 to 17C2 are diagrams illustrating an oblique angle determination based on captured images of the endoscopes.

FIGS. 18A1 to 18C2 are diagrams illustrating an oblique angle determination based on captured images of the endoscopes.

FIG. 19 is a diagram summarizing a relationship between the oblique angle of the endoscope and a circumferential orientation of the distal end portion.

FIGS. 20A1 to 20C2 are diagrams illustrating an oblique angle determination based on captured images of the endoscopes.

FIGS. 21A1 to 21C2 are diagrams illustrating an oblique angle determination based on captured images of the endoscopes.

FIG. 22 is a diagram summarizing a relationship between the oblique angle of the endoscope and the circumferential orientation of the distal end portion.

FIG. 23 is a plan view of a chart.

FIGS. 24A1 to 24C2 are diagrams illustrating an oblique angle determination based on captured images of the endoscopes.

FIG. 25 is a diagram summarizing a relationship between the oblique angle of the endoscope and the circumferential orientation of the distal end portion.

FIG. 26 is a diagram illustrating an oblique angle determination based on a captured image of the endoscope.

FIG. 27 is a diagram showing means for aligning the circumferential orientation of the distal end portion of the endoscope in a positive direction.

FIG. 28 is a plan view of a chart.

FIGS. 29A1 to 29C2 are diagrams illustrating an oblique angle determination based on captured images of the endoscopes.

FIGS. 30A1 to 30C2 are diagrams illustrating an outer diameter type determination based on captured images of the endoscopes.

FIGS. 31A1 to 31C2 are diagrams showing images captured by the endoscopes.

FIG. 32 is a flowchart illustrating the outer diameter type determination based on the captured images of the endoscopes.

FIGS. 33A1 to 33C2 are diagrams illustrating an outer diameter type determination based on captured images of the endoscopes.

FIGS. 34A1 to 34C2 are diagrams showing images captured by the endoscopes.

FIGS. 35A1 to 35C2 are diagrams illustrating an outer diameter type determination based on captured images of the endoscopes.

FIGS. 36A1 to 36C2 are diagrams showing images captured by the endoscopes.

FIGS. 37A1 to 37C2 are diagrams showing images captured by the endoscopes.

FIGS. 38A1 to 38C2 are diagrams illustrating an outer diameter type determination based on captured images of the endoscopes.

FIGS. 39A1 to 39C2 are diagrams showing images captured by the endoscopes.

FIGS. 40A1 to 40C2 are diagrams showing images captured by the endoscopes.

### DESCRIPTION OF THE PREFERRED EMBODIMENTS

Hereinafter, embodiments will be described with reference to the drawings as appropriate. First, the background leading to obtaining one aspect of the following embodiments will be described. In recent years, image processing has been performed on an endoscope image to acquire various pieces of information useful for diagnosis or the like, such as biological information of a subject, extraction of a lesion part, and differential information of the extracted lesion part. For example, as disclosed in JP2015-091467A and the like, there is known a technique of using an endoscope image to extract a low oxygen part with low oxygen saturation in a subject and displaying the low oxygen part on the endoscope image.

In the endoscope image used for image extraction of the low oxygen part in the subject, white balance adjustment needs to be performed severely in terms of color, that is, precise color adjustment in the color temperature needs to be performed. This is because the numerical values of the oxygen saturation fluctuate due to differences in color present in the endoscope image. Therefore, in order to perform precise white balance adjustment, it has been preferable to use a white balance adjustment chart for each of various endoscopes.

Therefore, in a case in which white balancing is performed using a dedicated chart for each of a plurality of types of endoscopes, charts respectively corresponding to the types of endoscopes are required, which may lead to the need to manage and handle the dedicated charts corresponding to various endoscopes, resulting in complicated operations. In addition, by using a plurality of charts, there is a possibility of errors arising from using a chart that does not correspond to a specific endoscope, which may result in white balance adjustments being made with incorrect settings.

Further, in order to perform precise white balance adjustment, it is important to appropriately adjust imaging conditions, such as a distance between the chart and a distal end surface of the endoscope provided with an objective lens of the endoscope, and an angle, in the imaging during the white balance adjustment. As described above, in various endoscopes, preparing a chart corresponding to each type of endoscope for white balance adjustment and imaging the chart after appropriately adjusting the imaging conditions may be time-consuming and lead to complicated adjustment.

Among various types of endoscopes, particularly in a rigid scope of a camera head system, a combination of the rigid scope, which is a scope, a camera head, and a light source is decided on immediately before use, and unlike a flexible scope, it is not possible to perform pre-shipment or pre-installation white balance adjustment (calibration). Therefore, before each procedure, a user, such as a doctor, needs to perform white balance adjustment for each of a plurality of rigid scopes to be used. Accordingly, in white balance adjustment for the rigid scope, particularly in white balance adjustment for the rigid scope capable of image extraction of the low oxygen part, as described above, it is necessary to prepare charts corresponding to various endoscopes and to individually image the chart under appropriate imaging conditions for each endoscope, which has been time-consuming.

Hereinafter, embodiments of a processor device that discriminates a type of endoscope and enables correction (image signal correction) of an image signal (image color), such as appropriate white balance adjustment or device variation correction, will be described.

### Types of Endoscopes

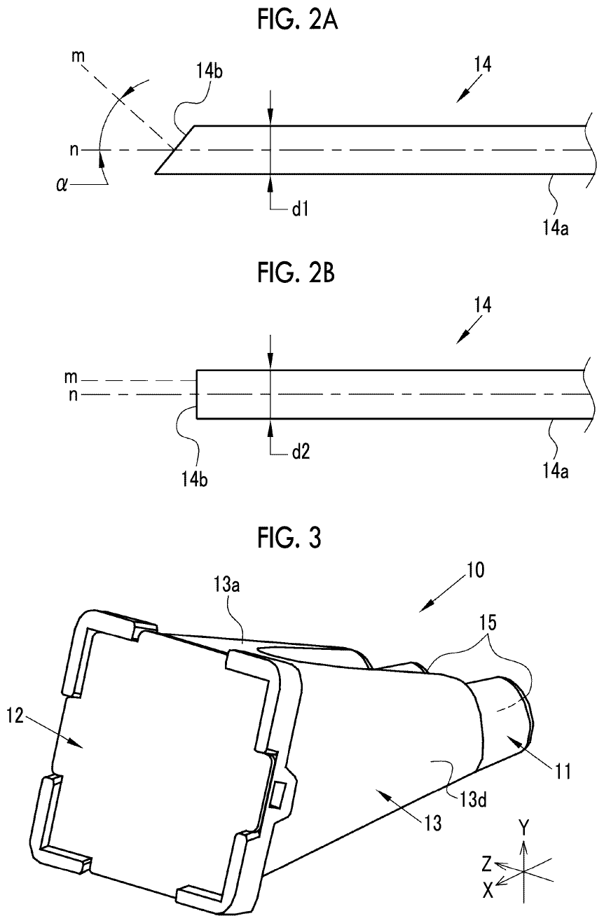

There are a plurality of types of endoscopes 14. For example, the types of endoscopes 14 are distinguished based on a difference in an outer diameter of a distal end portion 14a of the endoscope 14 and a difference in an angle of an imaging optical system in an optical axis direction with respect to an axial direction of the distal end portion 14a. Specifically, examples of the types based on the difference in the outer diameter include a large-diameter endoscope with a relatively large outer diameter of the distal end portion 14a and a small-diameter endoscope with a relatively small outer diameter of the distal end portion 14a. In a case in which the small-diameter endoscope and the large-diameter endoscope are distinguished from each other, the small-diameter endoscope is referred to as an endoscope 14A, and the large-diameter endoscope is referred to as an endoscope 14B. Additionally, examples of the types based on the difference in the angle of the imaging optical system in the optical axis direction include an oblique angle α (refer to FIGS. 2A and 2B), which is an angle formed by an optical axis direction m of the imaging optical system and an axial direction n of the distal end portion 14a, for example, 0 degrees, 30 degrees, 45 degrees, and the like. The endoscope 14 in which the oblique angle α is 0 degrees (the optical axis direction m and the axial direction n are parallel) is a direct-view scope, and the endoscope 14 in which the oblique angle α is 30 degrees, 45 degrees, or the like is an oblique-view scope. Further, the oblique-view scope may be referred to as a side-viewing scope or the like.

As shown in FIG. 2A, in the endoscope 14 as an oblique-view scope, the oblique angle α, which is an angle formed by the optical axis direction m of the imaging optical system and the axial direction n of the distal end portion 14a, is, for example, 30 degrees, 45 degrees, or the like. As shown in FIG. 2B, in the endoscope 14 as a direct-view scope, the optical axis direction m of the imaging optical system and the axial direction n of the distal end portion 14a are parallel to each other. The direct-view scope can obtain an endoscope image of a subject located in front of the distal end portion 14a in the axial direction, and the oblique-view scope can obtain an endoscope image of a subject located in front of a slightly angled side as viewed from the axial direction of the distal end portion 14a. In a case of the direct-view scope in which the optical axis direction m of the imaging optical system and the axial direction n of the distal end portion 14a are parallel to each other, the oblique angle α can be said to be 0 degrees. FIGS. 2A and 2B are views in which a direction in which a distal end surface 14b of the distal end portion 14a faces matches the optical axis direction m.

### Endoscope System

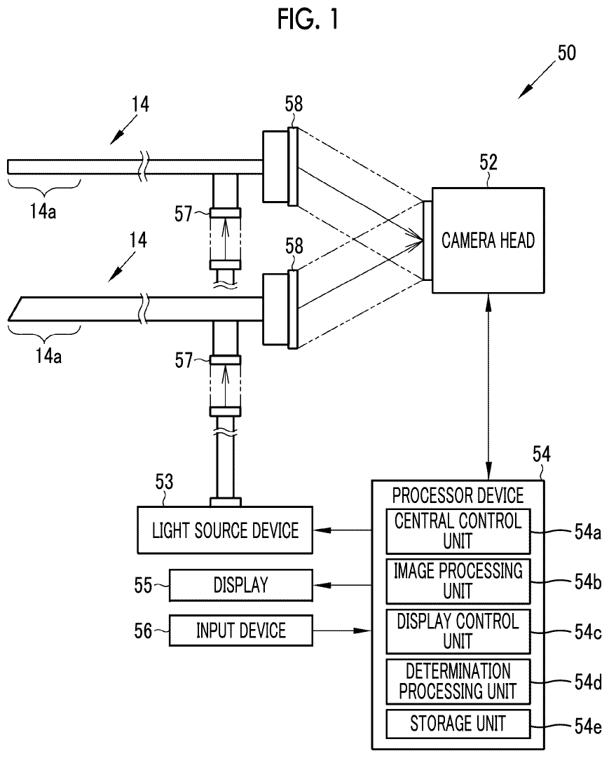

As shown in FIG. 1, the endoscope of the present embodiment is an intra-abdominal endoscope, which is a rigid scope (laparoscope) used in endoscopic surgery (laparoscopic surgery), and is a part of an intra-abdominal endoscope system 50 (hereinafter, referred to as an endoscope system 50).

The endoscope system 50 comprises the endoscope 14A and the endoscope 14B, a camera head 52, a light source device 53, a processor device 54, a display 55, and an input device 56. The endoscope system 50 is a system that uses the camera head 52. In the procedure, depending on the use or the like, any one of the endoscope 14A or the endoscope 14B is used by being coupled to the light source device 53 and the camera head 52 via a connector 57 for the light source device and a connector 58 for the camera head provided in each of the endoscope 14A and the endoscope 14B.

The endoscope 14A is, for example, a direct-view scope (refer to FIG. 2B), and the endoscope 14B is, for example, an oblique-view scope (refer to FIG. 2A). In addition, an outer diameter d2 of the distal end portion 14a of the endoscope 14A is 5 mm (refer to FIG. 2B), and an outer diameter d1 of the distal end portion 14a of the endoscope 14B is 10 mm (refer to FIG. 2A). In the following description, the endoscope 14A and the endoscope 14B are referred to as the endoscope 14 unless otherwise distinguished from each other.

The endoscope 14 is a rigid scope, is formed to be rigid and elongated, is inserted into a subject under examination, and is used for endoscopic surgery or the like. Before insertion into the subject under examination, image signal correction is performed on the endoscope 14A and the endoscope 14B, which are rigid scopes. The distal end portion 14a of the endoscope 14 is inserted into an endoscope color adjustment jig (hereinafter, referred to as an adjustment jig) 10 (refer to FIGS. 3 to 9) from an insertion hole 15 for image signal correction. Specifically, the endoscope 14A is inserted into a second insertion hole 15b, and the endoscope 14B is inserted into a first insertion hole 15a. After that, the distal end portion 14a of the endoscope 14 is advanced through an internal space 16 of the adjustment jig 10 to an appropriate position and images a mark 17 of a chart 18 provided on an inner surface 12b of a lid portion 12. In the following description, the first insertion hole 15a and the second insertion hole 15b are referred to as the insertion hole 15 unless otherwise distinguished from each other.

The imaging optical system for forming a subject image and an illumination optical system for irradiating the subject with illumination light are provided inside the endoscope 14. The light source device 53 generates illumination light toward the subject for imaging. The illumination light includes excitation light and the like. The light source device 53 includes a light source unit (not shown) consisting of a light source of, for example, a laser diode, a light emitting diode (LED), a xenon lamp, or a halogen lamp, and emits at least white illumination light or excitation light to be used to emit the white illumination light. Additionally, the light source unit includes, as needed, a phosphor that emits light by being irradiated with excitation light, an optical filter that adjusts a wavelength range, a spectrum, a light intensity, or the like of illumination light or excitation light, or the like. In addition, the light source unit emits light necessary for capturing an image, such as light with adjusted spectra, which is used to emphasize a specific tissue or the like in order to calculate biological information such as the oxygen saturation of hemoglobin included in the subject, or the like.

The camera head 52 comprises an imaging sensor or the like and images the subject. The processor device 54 comprises a central control unit 54a, an image processing unit 54b, a display control unit 54c, a determination processing unit 54d, and a storage unit 54e and performs system control of the endoscope system 50, image processing, and the like. The central control unit 54a performs system control of the endoscope system 50 in an integrated manner. The image processing unit 54b acquires the endoscope image acquired by the imaging sensor of the camera head 52 and generates various images. Additionally, the image processing unit 54b performs image signal correction and various types of processing related to the image signal correction. The display control unit 54c performs control to display a captured image 30 generated by the image processing unit 54b on the display 55 or the like. The determination processing unit 54d performs processing of determining the type of the endoscope 14. Specifically, the type of the endoscope 14 to be determined is the oblique angle α or the outer diameter of the distal end portion 14a. The central control unit 54a is a processor, and the central control unit 54a executes a predetermined program, thereby implementing the functions of the image processing unit 54b, the display control unit 54c, and the determination processing unit 54d. The storage unit 54e is, for example, storage means such as a memory or a hard disk. The display 55 is a display unit that displays the captured image 30 captured by the endoscope 14. The input device 56 is a console or the like and is an input device for setting input and the like to the processor device 54 or the like.

### Adjustment Jig

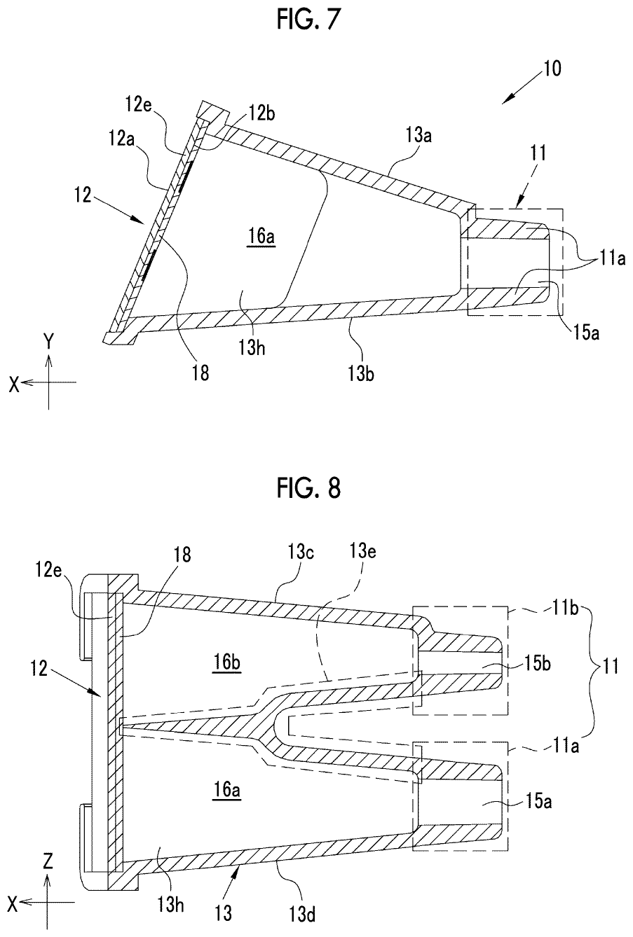

As shown in FIG. 3, the adjustment jig 10 is a tool used for color adjustment (image signal correction) of a plurality of types of endoscopes and comprises an insertion portion 11, the lid portion 12, and an outer peripheral portion 13. The outer peripheral portion 13 includes a left side surface portion 13d, a top surface portion 13a, a bottom surface portion 13b (refer to FIG. 7), and a right side surface portion 13c (refer to FIG. 8). The adjustment jig 10 has a substantially trapezoidal box shape that is composed of the insertion portion 11, the lid portion 12, and the outer peripheral portion 13, and includes the internal space 16 (refer to FIG. 7) inside. The internal space 16 may consist of internal spaces 16a and 16b (refer to FIG. 8), and in the following description, the internal spaces 16a and 16b are referred to as the internal space 16 unless otherwise distinguished from each other.

The color adjustment includes, for example, white balance adjustment (balance adjustment for white color). However, the present invention is not limited to the white balance adjustment, and the balance adjustment for colors other than white color may be performed. In addition, the color adjustment also includes, for example, image signal correction, such as device variation correction.

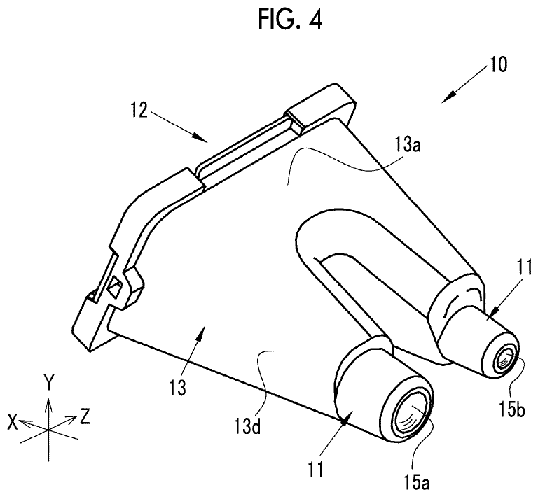

As shown in FIG. 4, the insertion portion 11 comprises two types of insertion holes 15 into which the distal end portion 14a of the endoscope 14 is inserted, that is, the first insertion hole 15a and the second insertion hole 15b. The first insertion hole 15a and the second insertion hole 15b have inner diameters different from each other, and the first insertion hole 15a has a larger inner diameter than the second insertion hole 15b. Additionally, the first insertion hole 15a and the second insertion hole 15b are provided such that the axial directions are parallel to the bottom surface portion 13b of the outer peripheral portion 13. The distal end portion 14a inserted in an insertion direction X from the insertion hole 15 is further advanced to an appropriate position of the internal space 16 and images the chart 18 (refer to FIG. 7) provided on an internal space 16 side of the lid portion 12 and, in some cases, an inner side surface 13h (refer to FIG. 7) of the outer peripheral portion 13. By using the captured image 30 obtained by imaging the chart 18, the type of the endoscope 14 is determined, and image signal correction is performed.

The insertion portion 11 may be provided with only one or three or more insertion holes 15. It is preferable that a plurality of insertion holes 15 have different inner diameters corresponding to outer diameters of the distal end portions 14a, which are different depending on the types of endoscopes 14 to be inserted. As a result, in a case in which the image signal correction is performed on each of the plurality of types of endoscopes 14, it is possible to prevent light from entering the internal space 16 from the insertion hole 15, and a guide function of advancing the distal end portion 14a to an appropriate position is exhibited, thereby allowing the chart 18 to be imaged appropriately and enabling the white balance to be appropriately adjusted.

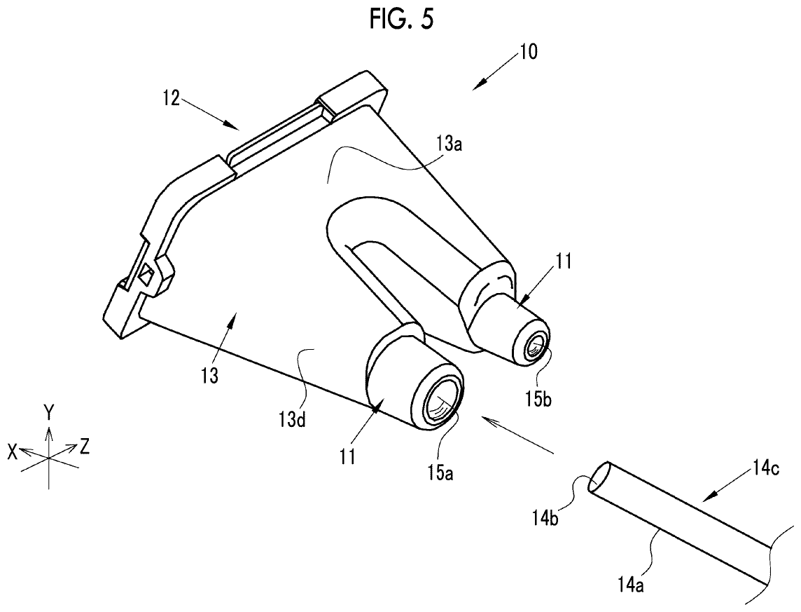

In addition, in order to ensure ease of insertion of the distal end portion 14a, a movable range for positioning adjustment of the distal end surface 14b (refer to FIG. 5) in a case of imaging the chart 18, and the like, it is preferable for the inner diameter of the insertion hole 15 to be as small as possible while allowing for a moderate gap between the distal end portion 14a and the insertion hole 15 in a case in which the distal end portion 14a is inserted into the insertion hole 15. Consequently, it is possible to achieve both ensuring a function of positioning adjustment of the distal end surface 14b by the insertion portion 11 guiding the insertion of the distal end portion 14a and restraining light from entering the internal space 16 from the gap between the distal end portion 14a and the insertion hole 15. For example, the inner diameter of the first insertion hole 15a at the inlet is 11 mm, and the inner diameter of the second insertion hole 15b at the inlet is 6 mm.

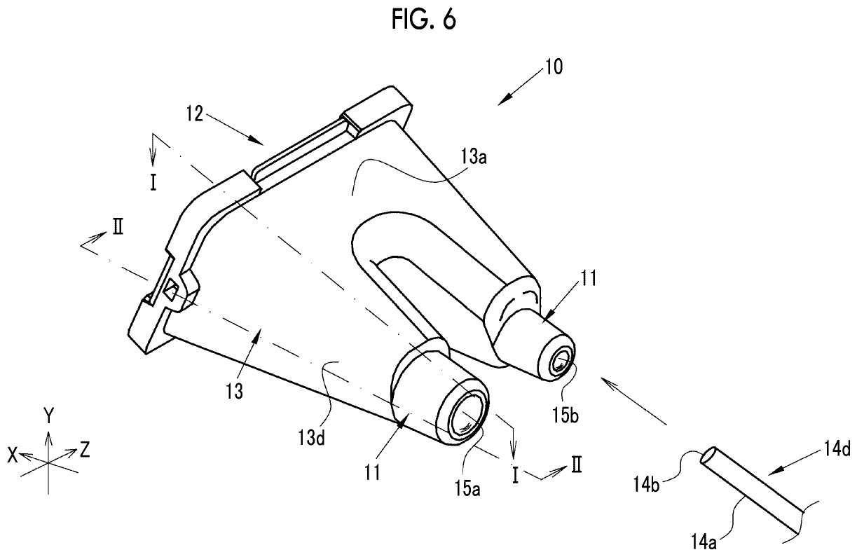

The distal end portion 14a is inserted into the insertion hole 15 selected in accordance with the outer diameter of the distal end portion 14a to be inserted. As shown in FIG. 5, in a case of a large-diameter endoscope in which the outer diameter dl (refer to FIG. 2A) of the distal end portion 14a is within a range of greater than 5 mm and 10 mm or less, the distal end portion 14a is inserted into the first insertion hole 15a. Additionally, as shown in FIG. 6, in a case of a small-diameter endoscope in which the outer diameter d2 (refer to FIG. 2B) of the distal end portion 14a is 5 mm or less, the distal end portion 14a is inserted into the second insertion hole 15b.

As shown in FIG. 7, the insertion portion 11 may be configured as a guide portion 11a. The guide portion 11a has a guide function in a case in which the user inserts the distal end portion 14a into the insertion hole 15. The guide portion 11a can be formed in a tubular shape in which a longitudinal direction is the insertion direction X. By forming the guide portion 11a in a tubular shape, in a case in which the distal end portion 14a is inserted by the user, a bottom portion of the distal end portion 14a, that is, a part of an outer surface or the like, is naturally inserted while coming into contact with a bottom portion of the tubular insertion hole 15, that is, a part of an inner surface or the like, which makes it easier to more appropriately insert the distal end portion 14a toward the lid portion 12. In addition, it is easier for the user to more easily dispose the distal end surface 14b of the endoscope 14 in the internal space 16 at an appropriate position.

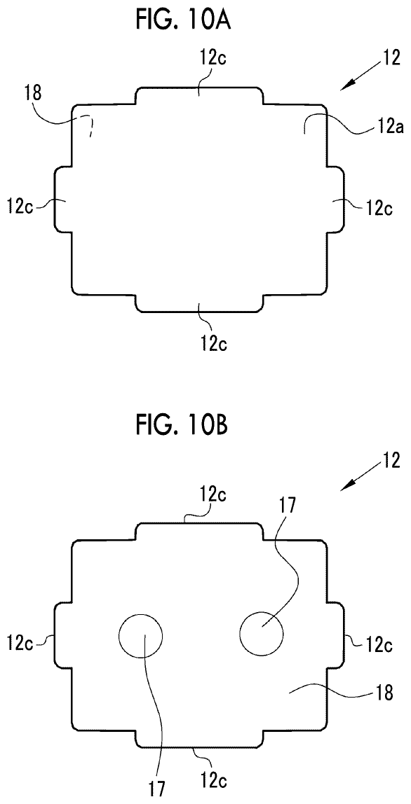

As shown in FIG. 8, the guide portion 11a and a guide portion 11b each may have a tapered shape in which the inner diameter thereof slightly increases from the inlet of the insertion hole 15 toward the lid portion 12 along the insertion direction X. The inner diameter of the insertion hole 15a at the inlet is 11 mm but is 12 mm at the outlet of the insertion hole 15a, which is located closest to the lid portion 12 along the insertion direction X in the guide portion 11a. Similarly, the inner diameter of the insertion hole 15b at the inlet is 6 mm but is 7 mm at the outlet of the insertion hole 15b, which is located closest to the lid portion 12 along the insertion direction X in the guide portion 11b. By providing the guide portion 11a and the guide portion 11b that are formed in a tapered shape along the insertion direction X, in a case in which the user inserts the distal end portion 14a into the insertion hole 15, it is easier to adjust and dispose the distal end portion 14a at a position where the mark 17 (refer to FIG. 10B) can be appropriately imaged, and it is possible to ensure the movable range for positioning adjustment of the distal end surface 14b of the endoscope 14 in a case in which the distal end portion 14a images the chart 18.

Additionally, in a case in which the insertion portion 11 includes a plurality of insertion holes 15 and the image of the inner side surface 13h of the outer peripheral portion 13 is not required for discriminating the type of the endoscope 14, the internal space 16 may be partitioned according to each of the insertion holes 15. In a case in which the plurality of insertion holes 15 are provided, the internal space 16 is partitioned by a partition 13e into an internal space 16a connected to the insertion hole 15a and an internal space 16b connected to the insertion hole 15b. Consequently, it is possible to prevent light from entering the internal space 16 from the insertion hole 15 into which the distal end portion 14a is not inserted. Therefore, by means of the internal space 16, which restrains the entry of external light, the chart 18 can be imaged under appropriate imaging conditions for image signal correction.

In addition, it is preferable that the partition 13e is shaped such that it can better block the entry of light into the insertion hole 15 into which the distal end portion 14a is inserted. Therefore, it is preferable that the partition 13e is in contact with the inner surface of the lid portion 12 that faces the internal space. Further, the partition 13e may be formed by extending the guide portion 11a into a tubular shape corresponding to each insertion hole 15.

As will be described in detail below, in a case in which the inner side surface 13h of the outer peripheral portion 13 of the adjustment jig 10 is actively imaged in addition to the chart 18 during imaging by the distal end portion 14a of the endoscope 14, or the like, the partition 13e may not be provided as mentioned above.

The lid portion 12 is disposed to face the insertion portion 11 in the insertion direction X of the distal end portion 14a in the insertion hole 15. The lid portion 12 functions as a constituent portion that constitutes a part of the housing of the adjustment jig 10 and as a chart holding portion that holds the chart 18. The lid portion 12 comprises a lid portion base 12e and the chart 18. In the lid portion base 12e, a surface located on the internal space 16 side in a case of configuring the adjustment jig 10 is referred to as the inner surface 12b, and a surface opposite to the inner surface 12b is referred to as an outer surface 12a (refer to FIG. 7). The inner surface 12b is provided with the chart 18. In the present embodiment, the lid portion 12 has a plate shape with a uniform thickness, and both surfaces are configured as planes. The shape of the lid portion base 12e or the lid portion 12 is not limited as long as the chart 18 provided on the lid portion base 12e is inclined with respect to a perpendicular plane perpendicular to the insertion direction X of the distal end portion 14a to allow the chart 18 to be held. Therefore, the lid portion base 12e or the lid portion 12 may have a plate shape with a non-uniform thickness or may not have a plate shape.

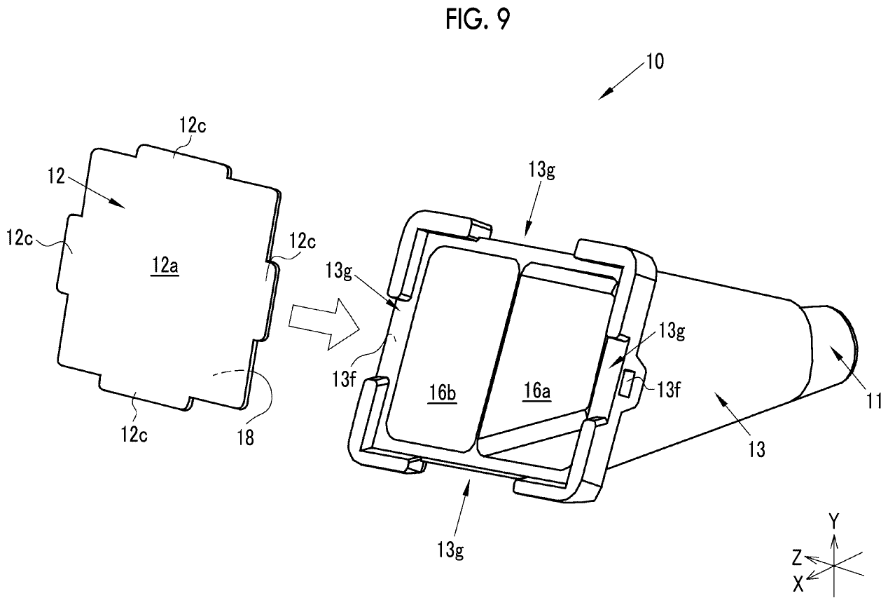

It is preferable that the lid portion 12 and the outer peripheral portion 13 are separably coupled to each other. As shown in FIG. 9, by releasing the lid portion 12 and the outer peripheral portion 13 from coupling, only the lid portion 12 can be removed from the adjustment jig 10. Therefore, the interior of the adjustment jig 10 can be cleaned, disinfected, and the like. Additionally, only the lid portion 12 can be cleaned, replaced, and the like, and the lid portion 12 provided with the chart 18 corresponding to the type of the endoscope 14 for which the image signal correction is to be performed can be selected from a plurality of lid portions 12 and can be used after the previously used lid portion 12 is replaced. The insertion portion 11 may be inseparably integrated with the outer peripheral portion 13 or may be separably coupled to the outer peripheral portion 13.

In a case in which the lid portion 12 and the outer peripheral portion 13 are separable from each other, it is preferable that the lid portion 12 and the outer peripheral portion 13 are configured to allow for easy separation and coupling. The lid portion 12 and the outer peripheral portion 13 may be separably coupled to each other by magnetic force and/or fitting. That is, the lid portion 12 and the outer peripheral portion 13 may be coupled to each other by using a magnet, or at least portions of the lid portion 12 and the outer peripheral portion 13 may be coupled to each other by the fitting of a protruding portion and a recessed portion or may be coupled to each other by using both the magnet and the fitting of the protruding portion and the recessed portion.

In the present embodiment, the outer peripheral portion 13 comprises a magnet insertion portion 13f, and a magnet (not shown) is fitted inside the magnet insertion portion 13f. The lid portion 12 includes a protruding portion 12c corresponding to at least the magnet insertion portion 13f, which has properties of being attracted by magnetic force to a part of a recessed portion 13g of the outer peripheral portion 13 that corresponds to the inside of the magnet insertion portion 13f. Therefore, the lid portion 12 and the outer peripheral portion 13 are easily coupled to each other by bringing the lid portion 12 closer to the outer peripheral portion 13 and are easily separated from each other by pulling the lid portion 12 away from the outer peripheral portion 13. In addition, since the protruding portion 12c is formed on the lid portion 12 and the recessed portion 13g corresponding to the protruding portion 12c is formed on the outer peripheral portion 13, the lid portion 12 and the outer peripheral portion 13 are easily coupled to each other at appropriate positions. Accordingly, the position of the chart 18 provided on the lid portion 12 can also be easily disposed at an appropriate position.

As shown in FIG. 10A, in the separated lid portion 12, the outer surface 12a constitutes an outer surface of the adjustment jig 10. As shown in FIG. 10B, the chart 18 is provided on a portion of the lid portion 12 that faces the internal space 16, and a surface of the chart 18 constitutes an inner surface of the adjustment jig 10. The mark 17 is provided on the surface of the chart 18. The mark 17 may be formed directly on the inner surface 12b of the lid portion base 12e, or the mark 17 may be formed on a substrate that constitutes the chart 18 and the chart 18 including the mark 17 may be disposed on the inner surface 12b. In this case, the shape of the chart 18 may be the same as or different from that of the lid portion 12. In the present embodiment, the chart 18 has a shape substantially identical to that of the lid portion 12 and has a rectangular shape provided with a protruding portion such as the protruding portion 12c.

The chart 18 is configured to enable appropriate correction of the image signal by being imaged by the endoscope 14 in a case in which image signal correction is performed. Therefore, in a case in which the distal end portion 14a is inserted from the insertion hole 15 and the chart 18 is imaged by the objective lens (not shown) provided in the distal end surface 14b of the distal end portion 14a, the chart 18 and the mark 17 are configured such that the mark 17 can be imaged in an appropriate state for the image signal correction of the endoscope 14. In the image signal correction, the image processing unit 54b creates an image signal correction parameter (correction parameter) based on the color of the imaged portion corresponding to the mark 17 in the captured image captured by the endoscope 14. Then, the image processing unit 54b uses the created image signal correction parameter to correct the image signal of the subsequent captured images 30. The objective lens is a part that constitutes the imaging optical system of the endoscope 14.

The chart 18 is disposed to be inclined with respect to the perpendicular plane perpendicular to the insertion direction X of the distal end portion 14a. Consequently, in a case in which the image signal correction is performed on each of the plurality of types of endoscopes 14, the chart 18 can be appropriately imaged, and the type of the endoscope 14 can be determined based on the captured image 30, and the image signal can be appropriately corrected. The perpendicular plane herein is a virtual plane.

In the present embodiment, the chart 18 is disposed to be inclined with respect to the perpendicular plane perpendicular to the insertion direction X of the distal end portion 14a, but the present invention is not limited thereto. The chart 18 may be disposed on the perpendicular plane perpendicular to the insertion direction X of the distal end portion 14a. In the determination of the type of the endoscope 14, which will be described below, any one of a configuration in which the chart 18 is disposed to be inclined with respect to the perpendicular plane perpendicular to the insertion direction X of the distal end portion 14a or a configuration in which the chart 18 is disposed on the perpendicular plane perpendicular to the insertion direction X of the distal end portion 14a is employed.

The color, shape, disposition, and the like of the mark 17 of the chart 18 are set in advance according to the type of the endoscope 14 on which the image signal correction is performed. Therefore, it is preferable that the mark 17 has a reference color suitable for correcting the image signal in each endoscope 14. The reference color is a color used to create white in the endoscope image. In the chart 18, it is preferable that a portion other than the mark 17 is formed in a color that makes the mark 17 easily recognizable. For example, in the chart 18, it is preferable that the portion other than the mark 17 has a color different from that of the mark 17 to make the mark 17 easily identifiable. Additionally, the number of marks 17 may be one or more. The number of marks 17 is decided on by the number, the type, or the like of the endoscope 14 that can be mounted on the adjustment jig 10. Even in the determination of the type of the endoscope 14, which will be described below, the number of marks 17 used is one or more depending on the number, the type, or the like of the endoscope 14 that can be mounted on the adjustment jig 10.

In addition, it is preferable that the mark 17 is shaped such that the shape of the mark 17 can be easily understood in a case in which the mark 17 is imaged in an appropriate state for the image signal correction of the endoscope 14, that is, in a state in which the distance between the distal end surface 14b and the chart 18 is appropriate. For example, the mark 17 may be shaped such that the shape, such as a circle, an ellipse, a triangle, a quadrangle, or a polygon, is easily recognizable. The shape also includes the size of the mark 17. Further, the shape need only be a shape that allows for appropriate image signal correction, and may be a filled shape, or a shape defined by a contour having a certain width without being filled. In the determination of the type of the endoscope 14, which will be described below, a circle is employed as the shape of the mark 17, but other shapes can also be employed as long as the determination is possible. Additionally, for example, the shape of the mark 17 may be a perfect circle or may not be a perfect circle. Further, in a case in which a plurality of marks 17 are provided, the plurality of marks 17 may have different sizes and shapes to make the differences recognizable or may be marked in a distinguishable manner.

Since the mark 17 has a specific shape, the user can determine whether or not the mark 17 is being imaged in an appropriate state based on the shape or the contour of the mark 17 by observing the display 55 on which the captured image 30 is displayed, in a case in which the distal end portion 14a is inserted into the insertion hole 15 and the chart 18 is imaged. The user can adjust the distance between the distal end surface 14b and the chart 18 such that the shape or the contour of the mark 17 is appropriate.

In this manner, in a case in which the distance between the distal end surface 14b and the chart 18, or the like is appropriate, the image signal correction may be performed using the video as it is, or a still image may be acquired and then the image signal correction may be performed using the still image.

In addition, it is preferable that the distal end portion 14a to be inserted into the adjustment jig 10 does not bend. Therefore, it is preferable that the endoscope 14 including the distal end portion 14a to be inserted into the insertion hole 15 is a rigid scope. In a case in which the endoscope 14 is a flexible scope, it is preferable that at least the distal end portion 14a of the scope does not bend.

### Adjustment of Insertion Amount of Endoscope into Adjustment Jig

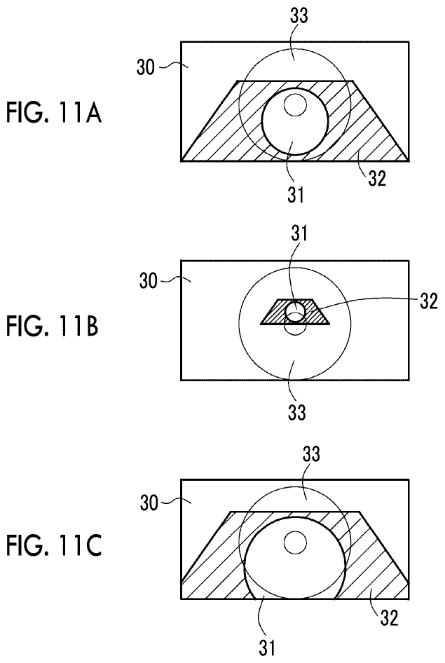

The insertion amount of the endoscope 14 into the adjustment jig 10 is adjusted using an imaging guide 33 displayed on the display 55. In a case in which the endoscope 14 is inserted into the insertion hole 15 of the adjustment jig 10 and the mark 17 provided on the chart 18 is imaged in a state in which the imaging guide 33 is displayed on the display 55, the captured image 30 is displayed on the display 55. Since the imaging guide 33 is displayed on the display 55, the captured image 30 of the mark 17 provided on the chart 18 is superimposed and displayed on the imaging guide 33 (FIGS. 11A to 11C).

In a case in which the chart 18 is disposed to be inclined with respect to the perpendicular plane perpendicular to the insertion direction X of the distal end portion 14a or in a case in which the endoscope 14 is an oblique-view scope, basically, the endoscope 14 images the chart 18 and the mark 17 from an oblique angle. Therefore, as shown in FIGS. 11A to 11C, the shape of an imaged portion (hereinafter, referred to as a chart portion) 32 corresponding to the chart 18 in the captured image 30 displayed on the display 55 and the shape of an imaged portion (hereinafter, referred to as a mark portion) 31 corresponding to the mark 17 appear distorted as if collapsed. That is, the shape of the chart portion 32 is a trapezoidal shape, and the shape of the mark portion 31 is an elliptical shape.

In a case in which the insertion of the distal end portion 14a of the endoscope 14 into the adjustment jig 10 is shallow, and the imaging is performed at a position farther from the chart 18, the chart portion 32 and the mark portion 31 in the captured image 30 appear small. On the other hand, in a case in which the insertion of the distal end portion 14a of the endoscope 14 is deep, and the imaging is performed at a position closer to the chart 18, the chart portion 32 and the mark portion 31 in the captured image 30 appear large. That is, the sizes of the chart portion 32 and the mark portion 31 in the captured image 30 displayed on the display 55 vary depending on the distance between the distal end portion 14a and the chart 18.

The shape and the size of the imaging guide 33 displayed on the display 55 are set in advance such that the mark portion 31 falls within the range of the imaging guide 33 in a case in which the insertion amount of the distal end portion 14a of the endoscope 14 is appropriate. The shape of the imaging guide 33 is a ring shape or a donut shape. Therefore, in a case in which the insertion amount of the distal end portion 14a of the endoscope 14 is appropriate, the mark portion 31 in the captured image 30 displayed on the display 55 falls within the range of the imaging guide 33 as shown in FIG. 11A. Here, in FIGS. 11A to 11C, in a case in which an outer periphery of the mark portion 31 falls within a region (ring portion) between an outer circumference and an inner circumference of the imaging guide 33, the mark portion 31 is considered to fall within the range of the imaging guide 33. On the other hand, in a case in which the insertion amount of the distal end portion 14a of the endoscope 14 is insufficient, as shown in FIG. 11B, the mark portion 31 in the captured image 30 protrudes beyond the inner side of the region of the imaging guide 33. Additionally, in a case in which the insertion amount of the distal end portion 14a of the endoscope 14 is too large, as shown in FIG. 11C, the mark portion 31 in the captured image 30 protrudes beyond the outer side of the region of the imaging guide 33.

As the image 30 used for the type determination of the endoscope 14, which will be described below, and the image signal correction, the captured image 30 shown in FIG. 11A, that is, the captured image 30 in which the mark portion 31 falls within the range of the imaging guide 33, is suitable. On the other hand, the captured image 30 shown in FIGS. 11B and 11C, that is, the captured image 30 in which the mark portion 31 protrudes beyond the region of the imaging guide 33 is not suitable. Therefore, the user adjusts the insertion amount of the distal end portion 14a of the endoscope 14 while observing the captured image 30 displayed on the display 55 such that the captured image 30 suitable for use in the oblique angle determination of the endoscope 14 and the image signal correction can be obtained.

### Oblique Angle Determination of Endoscope: First Embodiment

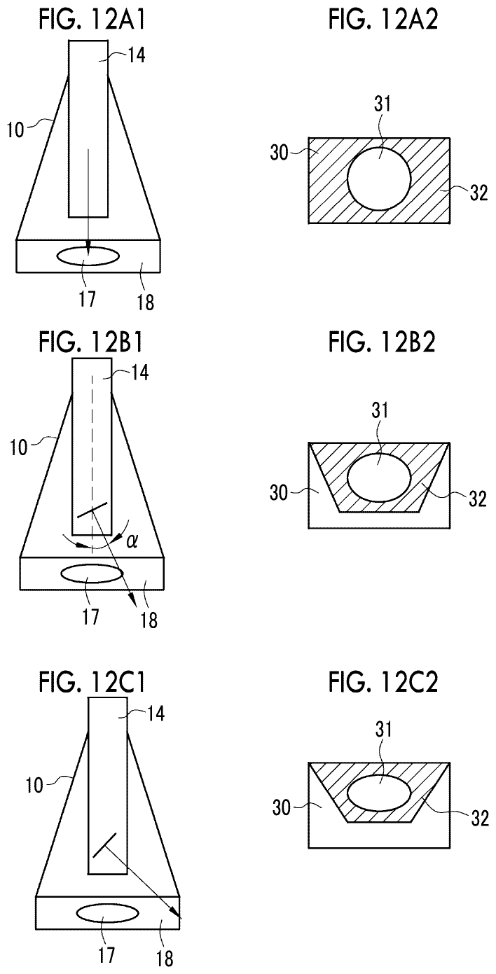

As a first embodiment, the oblique angle determination of the endoscope 14 in the endoscope system 50 will be described with reference to FIGS. 12A1 to 12C2. In the adjustment jig 10 here, the insertion portion 11 comprises one insertion hole 15. In addition, the chart 18 is disposed on the perpendicular plane perpendicular to the insertion direction X of the distal end portion 14a of the endoscope 14, and one mark 17 is provided in front of the distal end portion 14a. The mark 17 is circular (a perfect circle).

As shown in FIG. 12A1, in a case in which the endoscope 14 inserted into the insertion hole 15 of the insertion portion 11 of the adjustment jig 10 is, for example, a direct-view scope (oblique angle α=0 degrees), the chart 18 is disposed on the perpendicular plane perpendicular to the insertion direction X of the distal end portion 14a of the endoscope 14. Therefore, the endoscope 14 images the mark 17 provided on the chart 18 from the front. Accordingly, the shape of the mark portion 31 in the captured image 30 displayed on the display 55 is a perfect circle (FIG. 12A2). Additionally, as shown in FIG. 12B1, in a case in which the endoscope 14 inserted into the insertion hole 15 of the insertion portion 11 of the adjustment jig 10 is, for example, an oblique-view scope with an oblique angle α=30 degrees, the endoscope 14 images the mark 17 provided on the chart 18 at a 30-degree oblique angle. Therefore, the shape of the mark portion 31 in the captured image 30 is an ellipse with a relatively small degree of collapse (FIG. 12B2). Further, as shown in FIG. 12C1, in a case in which the endoscope 14 inserted into the insertion hole 15 of the insertion portion 11 of the adjustment jig 10 is, for example, an oblique-view scope with an oblique angle α=45 degrees, the endoscope 14 images the mark 17 provided on the chart 18 at a 45-degree oblique angle. Therefore, the shape of the mark portion 31 in the captured image 30 is an ellipse with a relatively large degree of collapse (FIG. 12C2). In FIGS. 12A1 to 12C2, the optical axis direction m is schematically shown as a direction in which a thin plate located in the distal end portion 14a faces. That is, in practice, the surface of the thin plate in the distal end portion 14a is the distal end surface 14b. The same applies to the drawings and descriptions, which will be described below.

In this manner, the shape of the mark portion 31 in the captured image 30 is correlated with the size of the oblique angle α of the endoscope 14. Specifically, the degree of collapse of the elliptical shape of the mark portion 31 in the captured image 30 displayed on the display 55 is proportional to the oblique angle α of the endoscope 14, that is, the greater the oblique angle α of the endoscope 14 is, the greater the degree of collapse is. Therefore, the oblique angle α of the endoscope 14 can be determined based on the shape of the mark portion 31 in the captured image 30 from such a correlation. Specifically, a relationship between the size of the oblique angle α of the endoscope 14 and the degree of collapse of the elliptical shape of the mark portion 31 in the captured image 30 (for example, the ratio of the length of the minor axis of the ellipse to the length of the major axis, or the like) is obtained in advance, and the relationship is stored in the storage unit 54e as a calculation expression, a table, or the like (hereinafter, referred to as a correspondence relationship table or the like). Meanwhile, the determination processing unit 54d of the processor device 54 causes the image processing unit 54b to perform image processing on the captured image 30 and obtains the degree of collapse of the elliptical shape of the mark portion 31 in the captured image 30. For example, the degree of collapse is converted into a feature amount. Then, the determination processing unit 54d refers to the correspondence relationship table or the like, compares the obtained degree of collapse (the measured feature amount of the shape) with the feature amount for comparison in the correspondence relationship table or the like, and obtains the size of the oblique angle α of the endoscope 14.

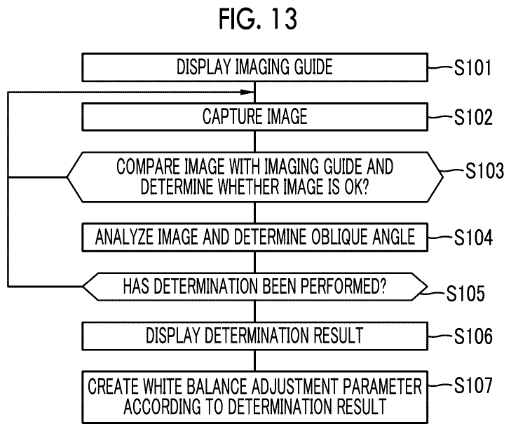

Next, an example of a flow of performing the type determination of the endoscope 14 will be described using the flowchart shown in FIG. 13. In this example, after the type determination of the endoscope 14 is performed, the creation of the image signal correction parameter is subsequently performed.

In a case in which the user operates the input device 56 to start the processing flow, the determination processing unit 54d of the processor device 54 causes the display control unit 54c to display the imaging guide 33 on the display 55 (step S101). Meanwhile, the user inserts the distal end portion 14a of the endoscope 14 into the insertion hole 15 of the insertion portion 11 of the adjustment jig 10, and the chart 18 and the mark 17 are imaged (step S102). The determination processing unit 54d may determine, based on an input image, that the distal end portion 14a of the endoscope 14 has been inserted into the insertion hole 15 and execute the imaging. In addition, in this case, it is preferable to select an appropriate captured image 30 from among the input images. In a case in which the endoscope 14 performs video capturing, frames of the video are the captured images 30, and at least one frame is used as the captured image 30. Further, in a case in which the endoscope 14 captures a still image in response to a still image acquisition instruction from the user, the still image captured in response to the still image acquisition instruction is used as the captured image 30.

The captured image 30 that has been captured is displayed on the display 55 by the display control unit 54c. In this case, since the imaging guide 33 is already displayed on the display 55, the captured image 30 and the imaging guide 33 are displayed on the display 55 in a superimposed manner.

The determination processing unit 54d determines whether or not the captured image 30 is suitable for the type determination of the endoscope 14 (step S103). Here, in a case in which the mark portion 31 in the captured image 30 protrudes beyond the region of the imaging guide 33, the captured image 30 is not suitable for the type determination of the endoscope 14 and cannot be employed. In such a case, the determination processing unit 54d determines that the captured image 30 is not suitable for the type determination of the endoscope 14. In a case in which the determination processing unit 54d determines that the captured image 30 that has been captured is not suitable for the type determination of the endoscope 14, the user is notified of the fact. Specifically, in a case in which the mark portion 31 in the captured image 30 protrudes beyond the region of the imaging guide 33, the determination processing unit 54d causes the display control unit 54c to perform error display on the display 55. Based on such a notification, the user adjusts the insertion amount of the distal end portion 14a of the endoscope 14 into the adjustment jig 10. After that, the imaging is performed again (step S102). Then, steps S102 and S103 are repeated until the captured image 30 suitable for the type determination of the endoscope 14 is obtained. The user may determine whether or not the captured image 30 is suitable for the type determination of the endoscope 14, instead of the determination processing unit 54d. In this case, the user repeats steps S102 and S103 until the captured image 30 suitable for the type determination of the endoscope 14 is obtained (the notification of the imaging state).

Then, in a case in which the captured image 30 suitable for the type determination of the endoscope 14 is obtained, that is, in a case in which the captured image 30 in which the mark portion 31 falls within the region of the imaging guide 33 is obtained, the determination processing unit 54d performs the type determination of the endoscope 14 (oblique angle determination) (step S104). Specifically, the determination processing unit 54d refers to the correspondence relationship table or the like stored in the storage unit 54e and determines the oblique angle of the endoscope 14 based on the shape of the mark portion 31 in the captured image 30. Then, in a case in which the determination processing unit 54d cannot determine the type (oblique angle) of the endoscope 14, the process returns from step S105 to step S102, and the chart 18 and the mark 17 are re-imaged. The case in which the type (oblique angle) of the endoscope 14 cannot be determined is, for example, a case in which there is no feature amount for comparison corresponding to the feature amount of the shape of the mark 17 in the captured image 30 even with reference to the correspondence relationship table or the like.

On the other hand, in step S105, in a case in which the determination processing unit 54d can determine the type (oblique angle) of the endoscope 14, the process proceeds to step S106, and the determination processing unit 54d causes the display control unit 54c to display the determination result, that is, the value of the oblique angle α, on the display 55 (outputs the value to the outside). Additionally, the determination processing unit 54d causes the image processing unit 54b to create the image signal correction parameter (correction parameter) corresponding to the determined type of the endoscope 14 (step S107).

In this manner, the oblique angle of the endoscope 14 can be determined based on the captured image 30 captured by the endoscope 14. Therefore, it is possible to create an image signal correction parameter corresponding to the type of the endoscope 14. As a result, image signal correction can be performed with greater accuracy.

In addition, by determining whether or not the captured image 30 is suitable for the type determination of the endoscope 14, the type of the endoscope 14 can be determined based on a more suitable captured image 30. As a result, image signal correction can be performed with greater accuracy.

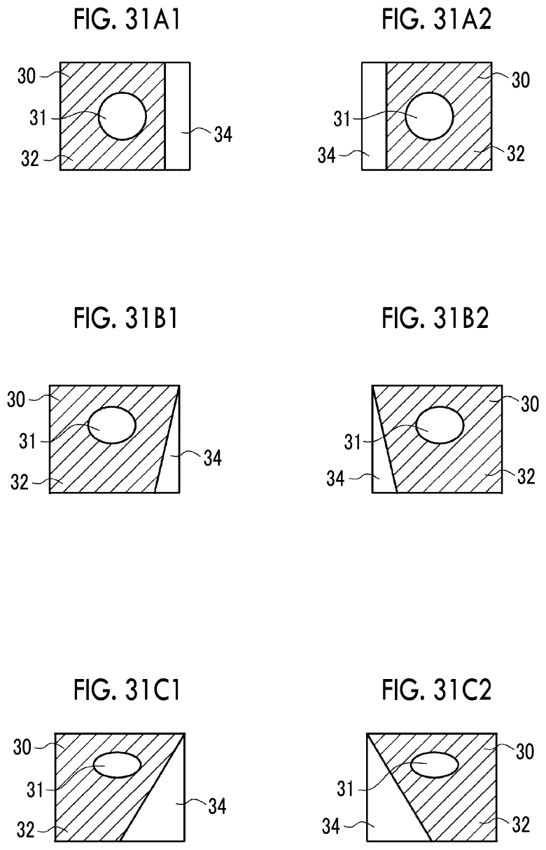

In the above description, the determination processing unit 54d determines the oblique angle of the endoscope 14 based on the shape of the mark portion 31 in the captured image 30, but the present invention is not limited thereto. For example, in a case in which the inner side surface 13h of the outer peripheral portion 13 is captured in the captured image 30, the oblique angle of the endoscope 14 may be determined based on the shape of an inner side surface portion 34 (refer to FIGS. 31A1 to 31C2) in the captured image 30, instead of the mark portion 31 or in addition to the mark portion 31. In this case as well, it is possible to create an image signal correction parameter corresponding to the type of the endoscope 14, and image signal correction can be performed with greater accuracy. The determination by the determination processing unit 54d can be performed using known image processing techniques, and the determination may be performed using a learning model in machine learning techniques, which has been trained on the position and the size of the mark portion 31, or, in a case of necessity, the inner side surface portion 34, in the captured image 30.

### Second Embodiment

In a second embodiment, in order to improve the accuracy of creating the image signal correction parameter of the endoscope 14, after the type determination of the endoscope 14 is performed, the captured image 30 more suitable for creating the image signal correction parameter is obtained using the imaging guide 33 corresponding to the type of the endoscope 14.

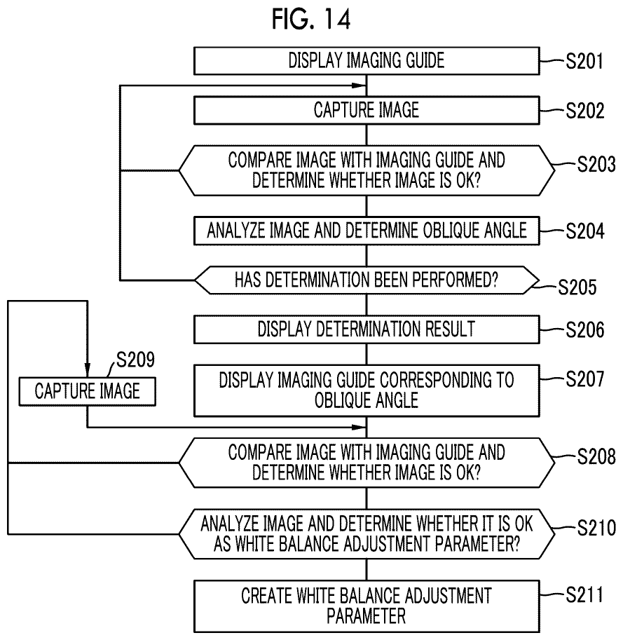

FIG. 14 shows an example of a flow of processing of obtaining the captured image 30 more suitable for creating the image signal correction parameter by using the imaging guide 33 corresponding to the type of the endoscope 14 after the type determination of the endoscope 14 is performed.

In a case in which the user operates the input device 56 to start the processing flow, the determination processing unit 54d of the processor device 54 causes the display control unit 54c to display the imaging guide 33 on the display 55 (step S201). Meanwhile, the user inserts the distal end portion 14a of the endoscope 14 into the insertion hole 15 of the insertion portion 11 of the adjustment jig 10, and the chart 18 and the mark 17 are imaged (step S202). The captured image 30 that has been captured is displayed on the display 55 by the display control unit 54c. In this case, the captured image 30 and the imaging guide 33 are displayed on the display 55 in a superimposed manner.

The determination processing unit 54d determines whether or not the captured image 30 is suitable for the type determination of the endoscope 14 (step S203). In a case in which the determination processing unit 54d determines that the captured image 30 is not suitable for the type determination of the endoscope 14, the user is notified of the fact. Based on the notification, the user adjusts the insertion amount of the distal end portion 14a of the endoscope 14 into the adjustment jig 10. After that, the imaging is performed again (step S202). Then, steps S202 and S203 are repeated until the captured image 30 suitable for the type determination of the endoscope 14 is obtained.

Then, in a case in which the captured image 30 suitable for the type determination of the endoscope 14 is obtained, that is, in a case in which the captured image 30 in which the mark portion 31 falls within the region of the imaging guide 33 is obtained, the determination processing unit 54d performs the type determination of the endoscope 14 (oblique angle determination) (step S204). In a case in which the determination processing unit 54d cannot determine the type (oblique angle) of the endoscope 14, the process returns from step S205 to step S202, and the chart 18 and the mark 17 are re-imaged.

On the other hand, in step S205, in a case in which the determination processing unit 54d can determine the type of the endoscope 14 (the type of the oblique angle), the process proceeds to step S206, and the determination processing unit 54d causes the display control unit 54c to display the determination result, that is, the value of the oblique angle α, on the display 55. In subsequent step S207, the determination processing unit 54d causes the display control unit 54c to display the imaging guide 33 corresponding to the type of the endoscope 14 on the display 55.

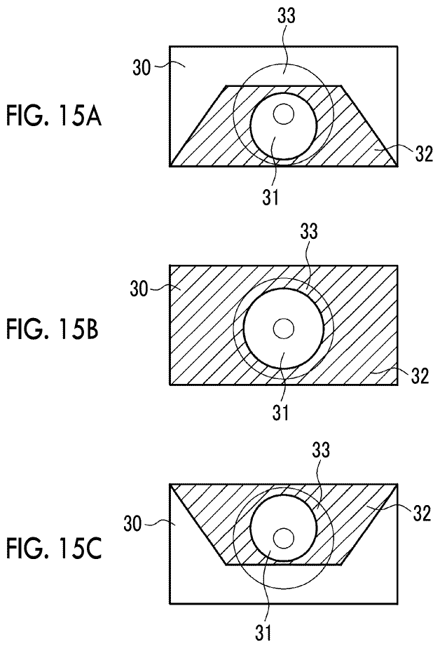

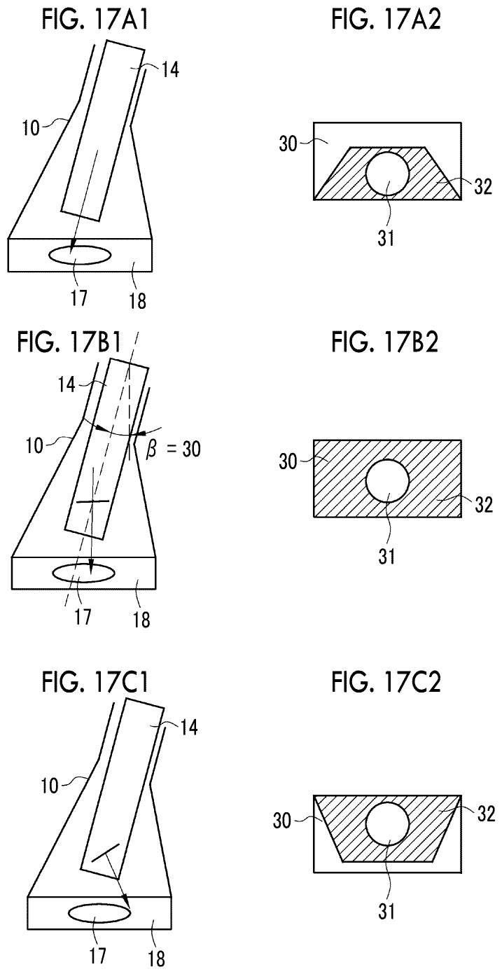

Here, the display of the imaging guide 33 corresponding to the type of the endoscope 14 will be described. FIGS. 15A to 15C are captured images 30 captured using the adjustment jig 10 in which the chart 18 is disposed to be inclined (for example, an inclination with an inclination angle β=22.5 degrees) with respect to the perpendicular plane perpendicular to the insertion direction X of the distal end portion 14a of the endoscope 14. The inclination angle β will be described in detail with reference to FIGS. 17A1 to 17C2. FIG. 15A is a captured image 30 in a case in which the endoscope 14 is a direct-view scope. FIG. 15B is a captured image 30 in a case in which the endoscope 14 is an oblique-view scope with an oblique angle α=30 degrees, and FIG. 15C is a captured image 30 in a case in which the endoscope 14 is an oblique-view scope with an oblique angle α=45 degrees.

As shown in FIG. 15A, since the oblique angle α: 0 degrees is a value sufficiently smaller than the inclination angle β: 22.5 degrees, the mark portion 31 in the captured image 30 is located slightly lower within the captured image 30. Additionally, as shown in FIG. 15B, since the oblique angle α: 30 degrees and the inclination angle β: 22.5 degrees are close values, the mark portion 31 in the image 30 is located near the center of the image 30. Further, as shown in FIG. 15C, since the oblique angle α: 45 degrees is a value sufficiently greater than the inclination angle β: 22.5 degrees, the mark portion 31 in the image 30 is located slightly above within the image 30. In this manner, the position of the mark portion 31 in the captured image 30 varies depending on the inclination angle β of the adjustment jig 10 to be used and the oblique angle α of the endoscope 14, and the position of the mark portion 31 in the captured image 30 is also decided on in a case in which the inclination angle β and the oblique angle α are decided on.

In order to address this, in the present embodiment, the imaging guide 33 corresponding to the type of the endoscope 14 (the type of the oblique angle) is stored in advance in the storage unit 54e for each adjustment jig 10. That is, data of the imaging guide 33 located on a lower side of the captured image so as to correspond to an oblique angle α of 0 degrees, data of the imaging guide 33 located at the center of the captured image so as to correspond to an oblique angle α of 30 degrees, and data of the imaging guide 33 located on an upper side of the captured image so as to correspond to an oblique angle α of 45 degrees are each stored in the storage unit 54e.

In step S207 of FIG. 14, the determination processing unit 54d reads the imaging guide 33 corresponding to the type of the endoscope 14 determined in step S205, as described above, from the storage unit 54e and causes the display control unit 54c to display the read imaging guide 33 on the display 55. As a result, the changed imaging guide 33 and the captured image 30 captured by the endoscope 14 are displayed on the display 55 in a superimposed manner.

The determination processing unit 54d determines whether or not the captured image 30 that has been captured can be used for creating the image signal correction parameter of the endoscope 14 (step S208). Here, in a case in which the mark portion 31 in the captured image 30 displayed on the display 55 protrudes beyond the region of the imaging guide 33, the captured image 30 cannot be employed. In such a case, the determination processing unit 54d determines that the captured image 30 that has been captured cannot be used for creating the image signal correction parameter of the endoscope 14. In a case in which the determination processing unit 54d determines that the captured image 30 cannot be used for creating the image signal correction parameter of the endoscope 14, the user is notified of the fact. Specifically, in a case in which the mark portion 31 in the captured image 30 protrudes beyond the region of the imaging guide 33, the determination processing unit 54d causes the display control unit 54c to perform error display on the display 55. Based on such a notification, the user adjusts the insertion amount of the distal end portion 14a of the endoscope 14 into the adjustment jig 10. After that, the imaging is performed again (step S209). Then, steps S208 and S209 are repeated until the captured image 30 that can be employed for the image signal correction of the endoscope 14 is obtained. Instead of the determination processing unit 54d, the user may check the display of the display 55 and determine whether or not the captured image 30 that has been captured can be used for creating the image signal correction parameter of the endoscope 14. In this case, the user repeats steps S208 and S209 until the captured image 30 that can be employed for the image signal correction of the endoscope 14 is obtained.

Then, in a case in which the captured image 30 that can be employed for creating the image signal correction parameter of the endoscope 14 is obtained, that is, in a case in which the captured image 30 in which the mark portion 31 falls within the region of the imaging guide 33 is obtained, the determination processing unit 54d determines whether or not the mark portion 31 in the captured image 30 can be employed for creating the image signal correction parameter of the endoscope 14 (step S210). Then, in a case in which the mark portion 31 cannot be employed, the process returns from step S210 to step S209, and the chart 18 and the mark 17 are re-imaged (step S209).

On the other hand, in step S210, in a case in which the mark portion 31 in the captured image 30 can be employed for creating the image signal correction parameter of the endoscope 14, the process proceeds to step S211, and the determination processing unit 54d causes the image processing unit 54b to create the image signal correction parameter.

In this manner, in the second embodiment, after the type determination of the endoscope 14 is performed, the captured image 30 more suitable for creating the image signal correction parameter is obtained using the imaging guide 33 corresponding to the type of the endoscope 14. Therefore, the accuracy of creating the image signal correction parameter of the endoscope 14 can be further improved.

### Third Embodiment

In a third embodiment, as the adjustment jig 10, an adjustment jig is used in which the chart 18 is disposed to be inclined with respect to the perpendicular plane perpendicular to the insertion direction X of the distal end portion 14a of the endoscope 14. That is, as in the adjustment jig 10 shown in FIG. 3, the insertion hole 15 is provided such that the axial direction is parallel to the bottom surface portion 13b of the outer peripheral portion 13. The inclination angle β with respect to the perpendicular plane is 22.5 degrees. In the third embodiment, the type of the endoscope 14 (the type of the oblique angle) is determined using such an adjustment jig 10.

Here, the relationship between the endoscope 14 and the adjustment jig 10 in a case in which the chart 18 and the like are imaged using the endoscope 14 will be described, and since the other parts are the same as those in the first embodiment or the second embodiment, the description thereof will be omitted.

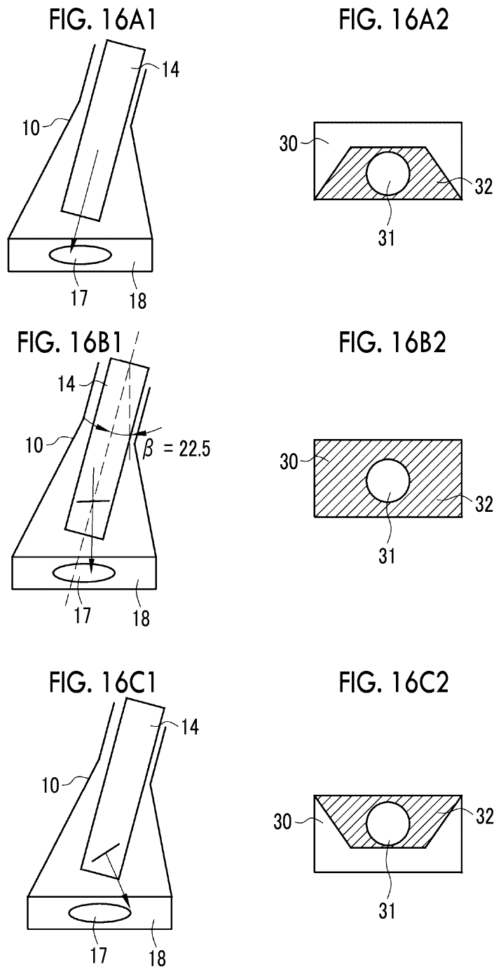

FIGS. 16A1, 16B1, and 16C1 are conceptual diagrams of a state in which the distal end portion 14a of the endoscope 14 is inserted into the adjustment jig 10. The endoscope 14 in FIG. 16A1 is a direct-view scope. In addition, the endoscope 14 in FIG. 16B1 is an oblique-view scope with an oblique angle α=30 degrees. Further, the endoscope 14 in FIG. 16C1 is an oblique-view scope with an oblique angle α=45 degrees. FIG. 16A2 shows the captured image 30 of the endoscope 14 shown in FIG. 16A1. Additionally, FIG. 16B2 shows the captured image 30 of the endoscope 14 shown in FIG. 16B1. Further, FIG. 16C2 shows the captured image 30 of the endoscope 14 shown in FIG. 16C1.

In the endoscope 14 shown in FIG. 16A1, an imaging angle (β-α) of the endoscope 14 with respect to the chart 18 is 22.5 degrees (=22.5-0). In addition, in the endoscope 14 shown in FIG. 16B1, the imaging angle (β-α) of the endoscope 14 with respect to the chart 18 is −7.5 degrees (=22.5-30). Further, in the endoscope 14 shown in FIG. 16C1, the imaging angle (β-α) of the endoscope 14 with respect to the chart 18 is −22.5 degrees (=22.5-45).

Meanwhile, the imaging angle in a case in which an adjustment jig (inclination angle β=0 degrees) is used as the adjustment jig 10 in which the chart 18 is disposed on the perpendicular plane perpendicular to the insertion direction X of the distal end portion 14a of the endoscope 14 is 0 degrees in the direct-view scope, 30 degrees in the oblique-view scope with an inclination angle of 30 degrees, and 45 degrees in the oblique-view scope with an inclination angle of 45 degrees. On the other hand, by using the adjustment jig 10 in which the chart 18 is disposed to be inclined with respect to the perpendicular plane perpendicular to the insertion direction X of the distal end portion 14a of the endoscope 14, the absolute value of the imaging angle of the endoscope 14 with respect to the chart 18 can be reduced. In a case in which the absolute value of the imaging angle decreases, the degree of collapse of the chart portion 32 and the mark portion 31 in the captured image 30 becomes smaller, and the area of the mark portion 31 in the captured image 30 increases. Therefore, the accuracy of creating the image signal correction parameter is improved. As a result, image signal correction can be performed with greater accuracy.

In the present embodiment, the inclination angle β is set to 22.5 degrees, but the present invention is not limited thereto. For example, the inclination angle β may be decided on according to the oblique angle of the endoscope 14 to be determined.

### Fourth Embodiment

In a fourth embodiment, the type of the endoscope 14 (the type of the oblique angle) is determined using, as the adjustment jig 10, an adjustment jig in which the chart 18 is disposed to be inclined by 30 degrees (inclination angle β=30 degrees) with respect to the perpendicular plane perpendicular to the insertion direction X of the distal end portion 14a of the endoscope 14.

Here, the relationship between the endoscope 14 and the adjustment jig 10 in a case in which the chart 18 and the like are imaged using the endoscope 14 will be described, and since the other parts are the same as those in the first embodiment or the second embodiment, the description thereof will be omitted.

In a case in which the captured image 30 is acquired for the type determination of the endoscope 14 or creating the image signal correction parameter, it may be necessary to match the circumferential orientation (the rotation angle or the insertion direction of the distal end portion 14a) of the distal end portion 14a of the endoscope 14 with a predetermined orientation (the rotation angle or the insertion direction) that has been decided on in advance. For example, the predetermined orientation that has been decided on in advance is defined as a positive direction, and a 180-degree rotated circumferential orientation is defined as a reverse direction.

FIGS. 17A1 to 17C2 show a case in which the circumferential orientation of the distal end portion 14a of the endoscope 14 is the positive direction, FIG. 17A1 is a conceptual diagram in a case in which the endoscope 14 is a direct-view scope, and FIG. 17A2 is an image 30 captured by the endoscope 14 in FIG. 17A1. Additionally, FIG. 17B1 is a conceptual diagram in a case in which the endoscope 14 is an oblique-view scope with an oblique angle α=30 degrees, and FIG. 17B2 is a captured image 30 captured by the endoscope 14 in FIG. 17B1. Further, FIG. 17C1 is a conceptual diagram in a case in which the endoscope 14 is an oblique-view scope with an oblique angle α=45 degrees, and FIG. 17C2 is a captured image 30 captured by the endoscope 14 in FIG. 17C1.

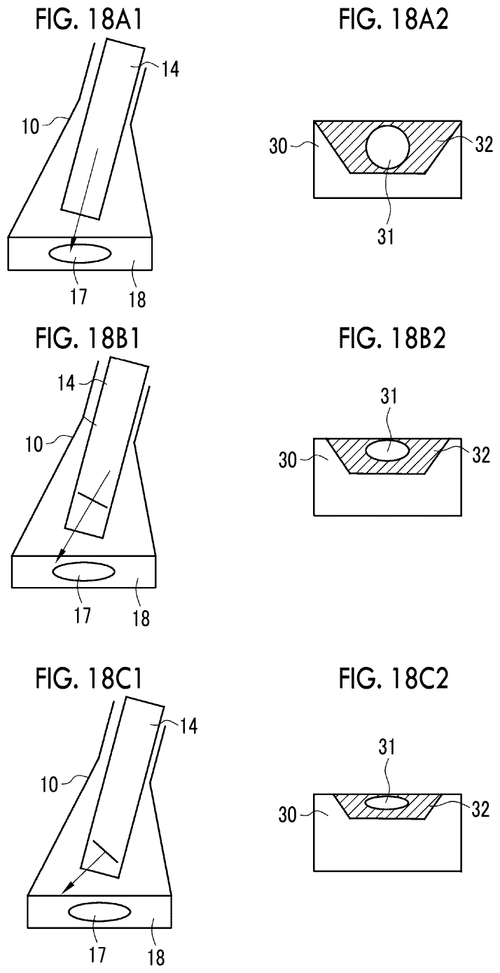

FIGS. 18A1 to 18C2 show a case in which the circumferential orientation of the distal end portion 14a of the endoscope 14 is in the reverse direction, FIG. 18A1 is a conceptual diagram in a case in which the endoscope 14 is a direct-view scope, and FIG. 18A2 is a captured image 30 captured by the endoscope 14 in FIG. 18A1. In addition, FIG. 18B1 is a conceptual diagram in a case in which the endoscope 14 is an oblique-view scope with an oblique angle α=30 degrees, and FIG. 18B2 is a captured image 30 captured by the endoscope 14 in FIG. 18B1. Further, FIG. 18C1 is a conceptual diagram in a case in which the endoscope 14 is an oblique-view scope with an oblique angle α=45 degrees, and FIG. 18C2 is a captured image 30 captured by the endoscope 14 in FIG. 18C1.

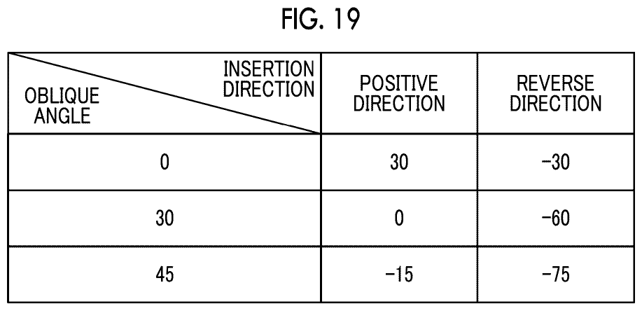

As shown in FIG. 19, in the endoscope 14 shown in FIG. 17A1, the imaging angle (β-α) of the endoscope 14 with respect to the chart 18 is 30 degrees (=30-0). Additionally, in the endoscope 14 shown in FIG. 17B1, the imaging angle (β-α) of the endoscope 14 with respect to the chart 18 is 0 degrees (=30-30). Further, in the endoscope 14 shown in FIG. 17C1, the imaging angle (β-α) of the endoscope 14 with respect to the chart 18 is −15 degrees (=30-45).

In addition, in the endoscope 14 shown in FIG. 18A1, the imaging angle (β-α) of the endoscope 14 with respect to the chart 18 is −30 degrees. Further, in the endoscope 14 shown in FIG. 18B1, the imaging angle (β-α) of the endoscope 14 with respect to the chart 18 is −60 degrees. Furthermore, in the endoscope 14 shown in FIG. 18C1, the imaging angle (β-α) of the endoscope 14 with respect to the chart 18 is −75 degrees.

As is clear from FIGS. 17A1 to 17C2 and FIGS. 18A1 to 18C2, the shapes of the chart portions 32 and the mark portions 31 of the six captured images 30 captured under the above-described conditions are all different. Therefore, such a relationship between the shape of the chart portion 32 and the mark portion 31 in the captured image 30 and the oblique angle α of the endoscope 14 is obtained in advance, and the relationship is stored in the storage unit 54e as the correspondence relationship table or the like. Then, by referring to the correspondence relationship table or the like to compare the shape of the chart portion and the mark portion in the correspondence relationship table or the like with the shape of the chart portion 32 and the mark portion 31 in the captured image 30 actually captured in the determination, the type (the type of the oblique angle) of the endoscope 14 inserted into the adjustment jig 10 can be determined.

Here, for comparison, a case is considered in which an adjustment jig is used as the adjustment jig 10 in which the chart 18 is disposed to be inclined by 22.5 degrees (inclination angle β=22.5 degrees) with respect to the perpendicular plane perpendicular to the insertion direction X of the distal end portion 14a of the endoscope 14. That is, a case is considered in which the adjustment jig 10 is used in which the inclination angle β differs by 7.5 degrees (=30-22.5).

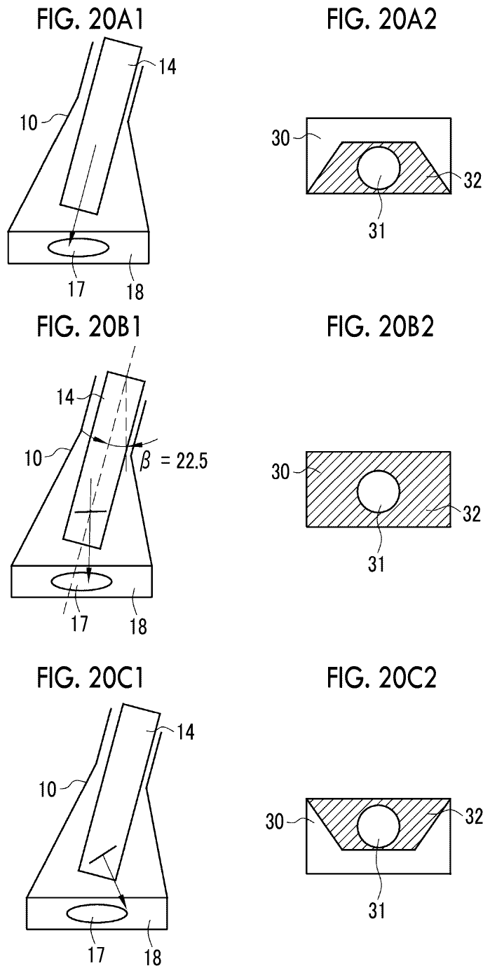

FIGS. 20A1 to 20C2 show a case in which the circumferential orientation of the distal end portion 14a of the endoscope 14 is in the positive direction, FIG. 20A1 is a conceptual diagram in a case in which the endoscope 14 is a direct-view scope, and FIG. 20A2 is a captured image 30 captured by the endoscope 14 in FIG. 20A1. Additionally, FIG. 20B1 is a conceptual diagram in a case in which the endoscope 14 is an oblique-view scope with an oblique angle α=30 degrees, and FIG. 20B2 is a captured image 30 captured by the endoscope 14 in FIG. 20B1. Further, FIG. 20C1 is a conceptual diagram in a case in which the endoscope 14 is an oblique-view scope with an oblique angle α=45 degrees, and FIG. 20C2 is a captured image 30 captured by the endoscope 14 in FIG. 20C1.

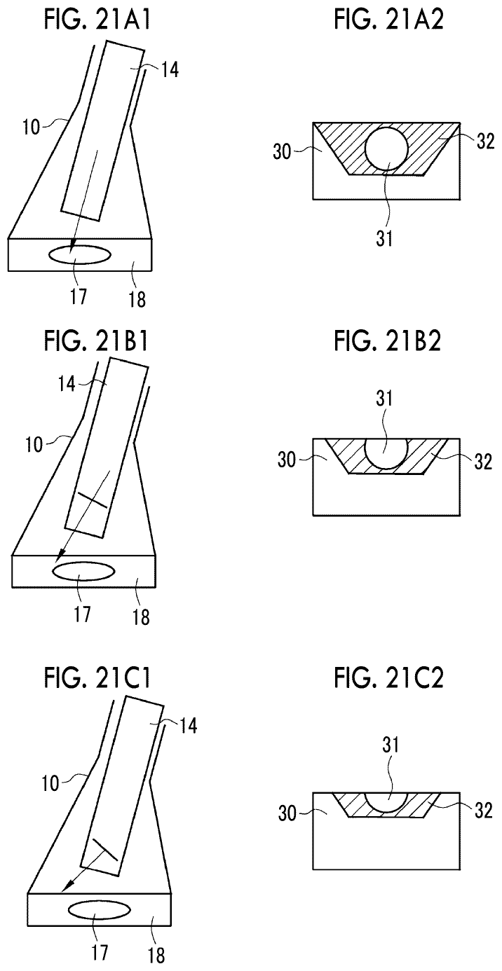

FIGS. 21A1 to 21C2 show a case in which the circumferential orientation of the distal end portion 14a of the endoscope 14 is in the reverse direction, FIG. 21A1 is a conceptual diagram in a case in which the endoscope 14 is a direct-view scope, and FIG. 21A2 is a captured image 30 captured by the endoscope 14 in FIG. 21A1. In addition, FIG. 21B1 is a conceptual diagram in a case in which the endoscope 14 is an oblique-view scope with an oblique angle α=30 degrees, and FIG. 21B2 is a captured image 30 captured by the endoscope 14 in FIG. 21B1. Further, FIG. 21C1 is a conceptual diagram in a case in which the endoscope 14 is an oblique-view scope with an oblique angle α=45 degrees, and FIG. 21C2 is a captured image 30 captured by the endoscope 14 in FIG. 21C1.

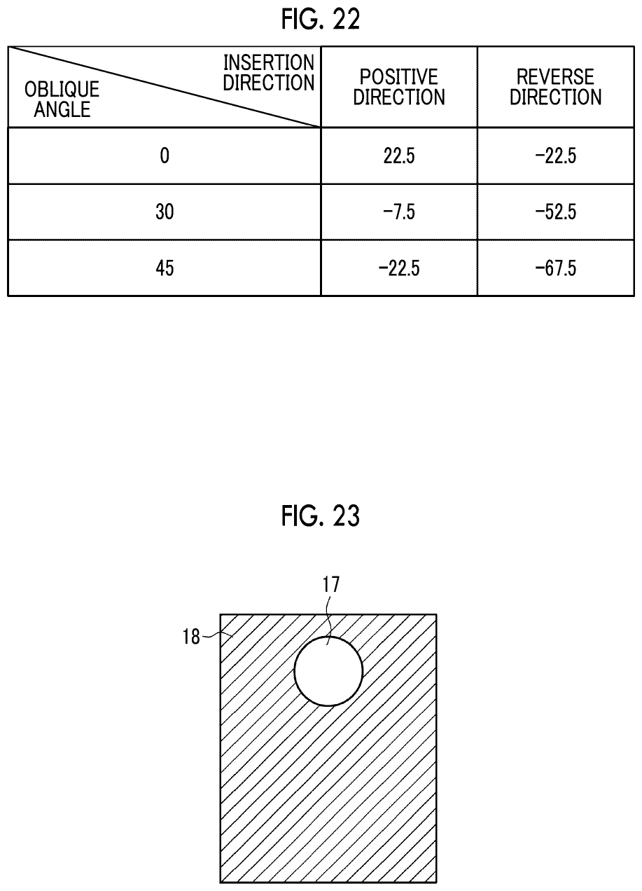

As shown in FIG. 22, in the endoscope 14 shown in FIG. 20A1, the imaging angle (β-α) of the endoscope 14 with respect to the chart 18 is 22.5 degrees (=22.5-0). Additionally, in the endoscope 14 shown in FIG. 20B1, the imaging angle (β-α) of the endoscope 14 with respect to the chart 18 is −7.5 degrees (=22.5-30). Further, in the endoscope 14 shown in FIG. 20C1, the imaging angle (β-α) of the endoscope 14 with respect to the chart 18 is −22.5 degrees (=22.5-45).

In addition, in the endoscope 14 shown in FIG. 21A1, the imaging angle (β-α) of the endoscope 14 with respect to the chart 18 is −22.5 degrees. Further, in the endoscope 14 shown in FIG. 21B1, the imaging angle (β-α) of the endoscope 14 with respect to the chart 18 is −52.5 degrees. Furthermore, in the endoscope 14 shown in FIG. 21C1, the imaging angle (β-α) of the endoscope 14 with respect to the chart 18 is −67.5 degrees.

As is clear from FIGS. 20A1 to 20C2 and FIGS. 21A1 to 21C2, among the six captured images 30 captured under the above-described conditions, the captured image 30 in FIG. 20C2 and the captured image 30 in FIG. 21A2 both having the same imaging angle of −22.5 degrees match each other. Therefore, there may be a case in which the relationship between the oblique angle α of the endoscope 14 and the chart portion 32 and the mark portion 31 of the captured image 30 does not correspond in a 1:1 manner, thereby making it impossible to determine the type of the endoscope 14 inserted into the adjustment jig 10 (the type of the oblique angle).

As described above, in a case in which the inclination angle β of the adjustment jig 10 is 22.5 degrees (½ of the inclination angle β=45 degrees), the adjustment jig 10 cannot be used for the type determination of the endoscope 14 through the image processing, but by setting the inclination angle β of the adjustment jig 10 to 30 degrees, the adjustment jig 10 can be used for the type determination of the endoscope 14 through the image processing. Therefore, it is possible to determine the type of the endoscope 14 based on the captured image 30 of the endoscope 14

### Fifth Embodiment

In each of the above-described embodiments, the type of the endoscope (the type of the oblique angle) is discriminated using the captured images of the chart 18 and the mark 17. Meanwhile, in a fifth embodiment, the type of the endoscope 14 (the type of the oblique angle) is discriminated using the shape of the inner side surface 13h of the outer peripheral portion 13 in the captured image 30. Here, as the chart 18, a chart is used in which the mark 17 is provided at a position displaced from the center, and the inner side surface 13h of the outer peripheral portion 13 is captured together with the chart 18 and the mark 17 in the captured image 30 by the endoscope 14.

Here, the relationship between the endoscope 14 and the adjustment jig 10 in a case in which the chart 18 and the like are imaged using the endoscope 14 will be described, and since the other parts are the same as those in the first embodiment or the second embodiment, the description thereof will be omitted.

FIG. 23 shows the chart 18 in the fifth embodiment. The mark 17 is provided at a position displaced from the center position of the chart 18. Additionally, as the adjustment jig 10, an adjustment jig is used in which the chart 18 is disposed to be inclined by, for example, 22.5 degrees (inclination angle β=22.5 degrees) with respect to the perpendicular plane perpendicular to the insertion direction X of the distal end portion 14a of the endoscope 14.

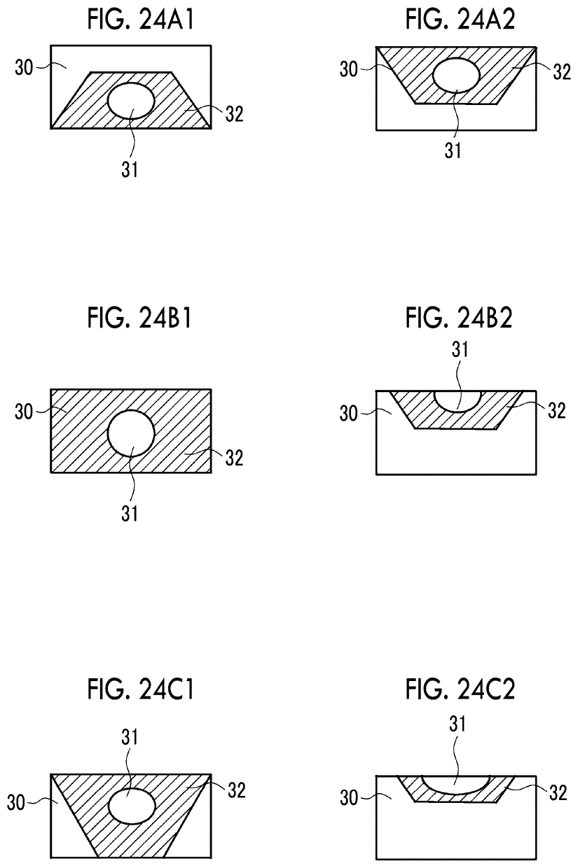

FIGS. 24A1 to 24C2 show captured images 30 by the endoscopes 14. FIG. 24A1 is a captured image 30 in a case in which a direct-view scope is used as the endoscope 14 and the circumferential orientation of the distal end portion 14a is in the positive direction. The imaging angle (β-α) is 22.5 degrees (=22.5-0). In addition, FIG. 24A2 is a captured image 30 in a case in which a direct-view scope is used as the endoscope 14 and the circumferential orientation of the distal end portion 14a is in the reverse direction. The imaging angle (β-α) is −22.5 degrees. Further, FIG. 24B1 is a captured image 30 in a case in which an oblique-view scope with an oblique angle α=30 degrees is used as the endoscope 14 and the circumferential orientation of the distal end portion 14a is in the positive direction. The imaging angle (β-α) is −7.5 degrees (=22.5-30). Additionally, FIG. 24B2 is a captured image 30 in a case in which an oblique-view scope with an oblique angle α=30 degrees is used as the endoscope 14 and the circumferential orientation of the distal end portion 14a is in the reverse direction. The imaging angle (β-α) is −52.5 degrees. Further, FIG. 24C1 is a captured image 30 in a case in which an oblique-view scope with an oblique angle α=45 degrees is used as the endoscope 14 and the circumferential orientation of the distal end portion 14a is in the positive direction. The imaging angle (β-α) is −22.5 degrees (=22.5-45). Furthermore, FIG. 24C2 is a captured image 30 in a case in which an oblique-view scope with an oblique angle α=45 degrees is used as the endoscope 14 and the circumferential orientation of the distal end portion 14a is in the reverse direction. The imaging angle (β-α) is −67.5 degrees.

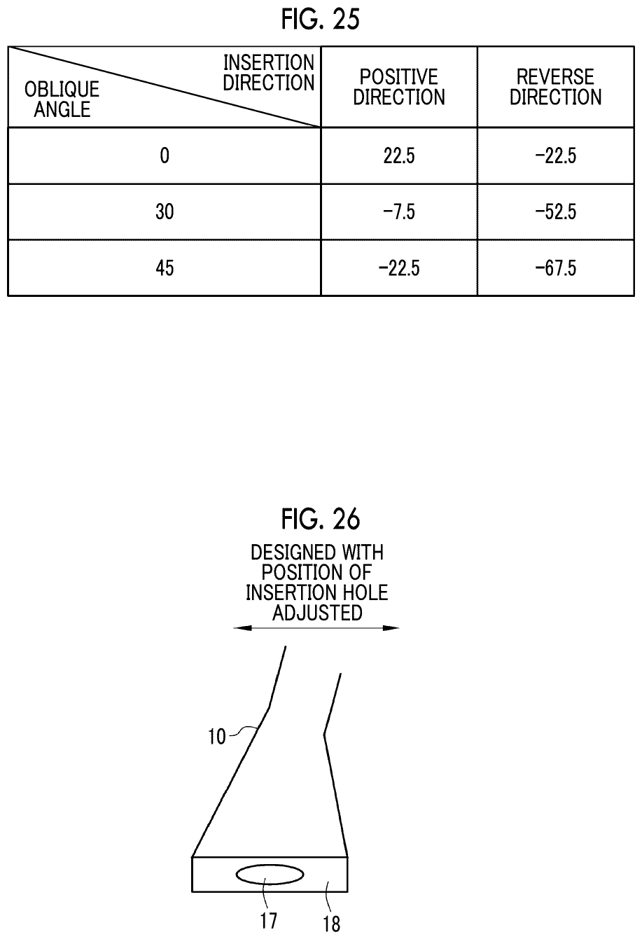

FIG. 25 collectively shows imaging angles of the captured images 30. As is clear from FIG. 25, the imaging angle (−22.5 degrees) of the endoscope 14 in FIG. 24C1 and the imaging angle (−22.5 degrees) of the endoscope 14 in FIG. 24A2 match each other. Therefore, the shape and the size of the mark portion 31 in the captured image 30 in FIG. 24C1 and the shape and the size of the mark portion 31 in the captured image 30 in FIG. 24A2 match each other. Therefore, it is difficult to discriminate the type of the endoscope 14 based solely on the mark portion 31.

Meanwhile, in the fifth embodiment, since a chart is used as the chart 18 in which the mark 17 is provided at a position displaced from the center, the inner side surface 13h of the outer peripheral portion 13 is captured in the captured image 30 together with the chart 18 and the mark 17. In the captured images 30 for each combination of the oblique angle α and the circumferential orientation of the distal end portion 14a, the shape and the size of the inner side surface portion 34 are different. Therefore, it is possible to distinguish between the endoscope 14 that has captured the captured image 30 in FIG. 24C1 and the endoscope 14 that has captured the captured image 30 in FIG. 24A2, which has made it difficult to discriminate the type of the endoscope 14 based solely on the mark portion 31 in the captured image 30.

Other endoscopes 14 with different imaging angles can be discriminated based on the shape and the size of the mark portion 31 in the captured image 30.

In addition, the adjustment jig 10 of the present embodiment is designed with the position of the insertion hole 15 adjusted in advance such that the mark 17 can be imaged by the distal end portion 14a of the endoscope 14 (FIG. 26).

### Sixth Embodiment

In each of the above-described embodiments, in a case in which the distal end portion 14a of the endoscope 14 is inserted into the insertion hole 15 of the adjustment jig 10, the distal end portion 14a is rotatable in the circumferential direction thereof and can be inserted not only in a state in the positive direction and can be inserted but also in, for example, a state in the reverse direction. Meanwhile, in a sixth embodiment, means for aligning the circumferential orientation of the distal end portion 14a with the orientation in the positive direction is provided.

As shown in FIG. 27, as means 20 for aligning the circumferential orientation of the distal end portion 14a with the orientation in the positive direction, for example, a configuration is available in which alignment marks 20a and 20b are attached to the vicinity of the inlet of the insertion hole 15 of the adjustment jig 10 and the distal end portion 14a of the endoscope 14, and the circumferential orientation of the distal end portion 14a is set to the orientation in the positive direction in a case in which the alignment marks 20a and 20b are aligned with each other. In this case, the circumferential orientation of the distal end portion 14a can be set to the positive direction easily and accurately at a low cost.

The means for aligning the circumferential orientation of the distal end portion 14a with the orientation in the positive direction is not limited to the alignment marks, and other configurations may be available as long as the circumferential orientation of the distal end portion 14a can be aligned with the orientation in the positive direction.

### Seventh Embodiment

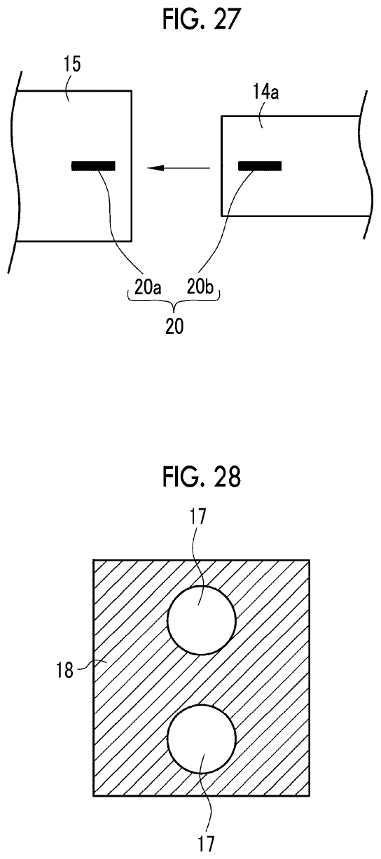

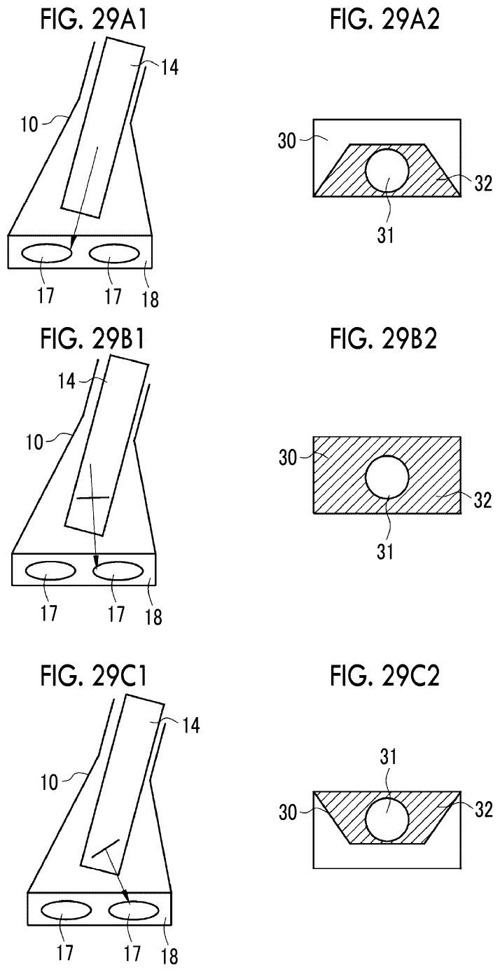

In each of the above-described embodiments, the chart 18 has been used in which only one mark 17 is provided. Meanwhile, in a seventh embodiment, the chart 18 is used in which a plurality of marks 17 are provided. Here, the relationship between the endoscope 14 and the adjustment jig 10 in a case in which the chart 18 and the like are imaged using the endoscope 14 will be described, and since the other parts are the same as those in the first embodiment or the second embodiment, the description thereof will be omitted. FIG. 28 shows a chart 18 in which two marks 17 are provided. The two marks 17 are provided on the chart 18 to be parallel to the right side surface portion 13c or the left side surface portion 13d. Additionally, FIGS. 29A1, 29B1, and 29C1 are conceptual diagrams of a state in which the distal end portion 14a of the endoscope 14 is inserted into the adjustment jig 10. The endoscope 14 in FIG. 29A1 is a direct-view scope. In addition, the endoscope 14 in FIG. 29B1 is an oblique-view scope with an oblique angle α=30 degrees. Further, the endoscope 14 in FIG. 29C1 is an oblique-view scope with an oblique angle α=45 degrees. Additionally, FIG. 29A2 shows the captured image 30 of the endoscope 14 shown in FIG. 29A1. Further, FIG. 29B2 shows the captured image 30 of the endoscope 14 shown in FIG. 29B1. Furthermore, FIG. 29C2 shows the captured image 30 of the endoscope 14 shown in FIG. 29C1.

The adjustment jig 10 is set up with the chart 18 that includes two marks 17. Among the two marks 17, one mark 17 located in front of the distal end portion 14a in the axial direction n is an imaging target mainly in a case in which the endoscope 14 is a direct-view scope (FIG. 29A1). The other mark 17 is an imaging target mainly in a case in which the endoscope 14 is an oblique-view scope (FIGS. 29B1 and 29C1).

In this manner, the mark 17 as the imaging target can be changed according to the value of the oblique angle α of the endoscope 14, and the mark 17 can be imaged from near the front in any case of the direct-view scope or the oblique-view scope. Therefore, in any case of the direct-view scope or the oblique-view scope, the imaging angle of the mark 17 can be reduced, and the degree of collapse of the shape of the mark portion 31 and the chart portion 32 in the captured image 30 captured by the endoscope 14 can be reduced. Therefore, the area of the mark portion 31 in the captured image 30 increases, and the accuracy of creating the image signal correction parameter can be improved.

### Eighth Embodiment

Next, the outer diameter type determination of the endoscope 14 will be described as an eighth embodiment. In a case in which the outer diameter type determination is performed, an adjustment jig is used as the adjustment jig 10 in which a plurality of insertion holes 15 are provided. In the present embodiment, since the determination of the large diameter and the small diameter is performed for the outer diameter dimension of the distal end portion 14a of the endoscope 14, two insertion holes 15a and 15b with different inner diameters are provided. The insertion holes 15a and 15b are provided at substantially the center position between the top surface portion 13a and the bottom surface portion 13b of the outer peripheral portion 13. In addition, the chart 18 of the adjustment jig 10 is disposed on the perpendicular plane perpendicular to the insertion direction X of the distal end portion 14a of the endoscope 14 (inclination angle β=0 degrees). Further, as the adjustment jig 10, an adjustment jig is used in which the partition 13e is not provided, and the inner side surface 13h of the outer peripheral portion 13 is also captured in the captured image 30 together with the mark 17.

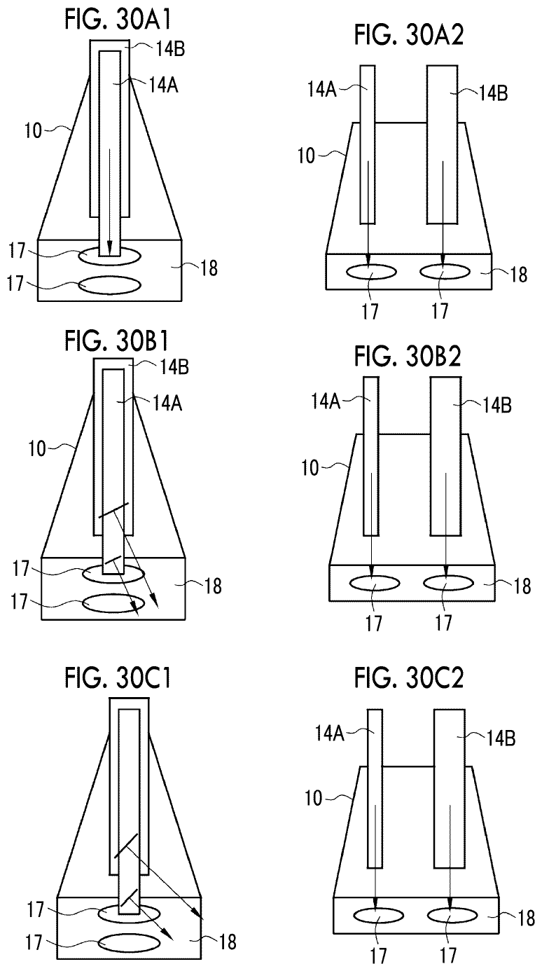

FIGS. 30A1 to 30C2 are conceptual diagrams of a state in which the distal end portion 14a of the endoscope 14 is inserted into the adjustment jig 10 in order to perform the outer diameter type determination of the endoscope 14. In FIGS. 30A1 to 30C2, two types of endoscopes 14 (for example, an endoscope with an outer diameter of 5 mm and an endoscope with an outer diameter of 10 mm) are depicted as being simultaneously inserted, but in practice, the outer diameter type determination is performed one by one. In a case in which the endoscope 14 with an outer diameter of 5 mm and the endoscope 14 with an outer diameter of 10 mm are described in a distinguished manner, the endoscope 14 with an outer diameter of 5 mm is referred to as the endoscope 14A, and the endoscope 14 with an outer diameter of 10 mm is referred to as the endoscope 14B.

FIGS. 30A1, 30B1, and 30C1 are conceptual diagrams of the adjustment jig 10 as viewed from a Z direction (refer to FIG. 3), and FIGS. 30A2, 30B2, and 30C1 are conceptual diagrams of the adjustment jig 10 as viewed from a Y direction (refer to FIG. 3). The endoscopes 14A and 14B in FIGS. 30A1 and 30A2 are direct-view scopes. Additionally, the endoscopes 14A and 14B in FIGS. 30B1 and 30B2 are oblique-view scopes with an oblique angle α=30 degrees. Further, the endoscopes 14A and 14B in FIGS. 30C1 and 30C2 are oblique-view scopes with an oblique angle α=45 degrees. Here, the Z direction is a direction that is perpendicular to the insertion direction X and that matches an arrangement direction of the two insertion holes 15a and 15b, and the Y direction is a direction perpendicular to a plane including the insertion direction X and the Z direction.

The captured image 30 of the endoscope 14B shown in FIGS. 30A1 and 30A2 is shown in FIG. 31A1. In addition, the captured image 30 of the endoscope 14A shown in FIGS. 30A1 and 30A2 is shown in FIG. 31A2. Further, the captured image 30 of the endoscope 14B shown in FIGS. 30B1 and 30B2 is shown in FIG. 31B1. Additionally, the captured image 30 of the endoscope 14A shown in FIGS. 30B1 and 30B2 is shown in FIG. 31B2. Further, the captured image 30 of the endoscope 14B shown in FIGS. 30C1 and 30C2 is shown in FIG. 31C1. Furthermore, the captured image 30 of the endoscope 14A shown in FIGS. 30C1 and 30C2 is shown in FIG. 31C2.

As is clear from the six captured images 30 shown in FIGS. 31A1 to 31C2, the shapes of the mark portion 31 and the inner side surface portion 34 of the outer peripheral portion 13 in the captured image 30 are all different. More specifically, the degree of collapse of the mark portion 31 and the position of the inner side surface portion 34 are different. Therefore, a relationship between the distinction of the outer diameter of the endoscope 14 and the oblique angle α, and the shapes of the mark portion 31 and the inner side surface portion 34 of the outer peripheral portion 13 in the captured image 30 is obtained in advance, and the relationship is stored in the storage unit 54e as the correspondence relationship table or the like. Then, by referring to the correspondence relationship table or the like to compare the shapes (for example, the measured feature amounts of the shapes) of the mark portion 31 and the inner side surface portion 34 of the outer peripheral portion 13 in the captured image 30 with the shapes (for example, the feature amounts for comparison) of the mark portion and the inner side surface portion in the correspondence relationship table or the like, the outer diameter type determination of the endoscope 14 inserted into the adjustment jig 10 can be performed. Therefore, it is possible to create an image signal correction parameter corresponding to the type of the endoscope 14. As a result, image signal correction can be performed with greater accuracy.

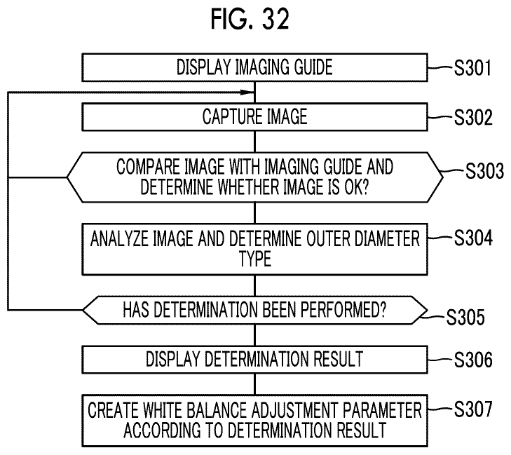

Next, an example of a flow of performing the outer diameter type determination of the endoscope 14 will be described using the flowchart shown in FIG. 32. In this example, after the outer diameter type determination of the endoscope 14 is performed, the creation of the image signal correction parameter is subsequently performed.

In a case in which the user operates the input device 56 to start the processing flow, the determination processing unit 54d of the processor device 54 causes the display control unit 54c to display the imaging guide 33 on the display 55 (step S301). Meanwhile, the user inserts the distal end portion 14a of the endoscope 14 into the insertion hole 15 of the insertion portion 11 of the adjustment jig 10, and the chart 18, the mark 17, and the inner side surface 13h of the outer peripheral portion 13 are imaged (step S302). The determination processing unit 54d may determine, based on the input image, that the distal end portion 14a of the endoscope 14 has been inserted into the insertion hole 15 and execute the imaging. In addition, in this case, it is preferable to select an appropriate captured image 30 from among the input images. In a case in which the endoscope 14 performs video capturing, frames of the video are the captured images 30, and at least one frame is used as the captured image 30. Further, in a case in which the endoscope 14 captures a still image in response to a still image acquisition instruction from the user, the still image captured in response to the still image acquisition instruction is used as the captured image 30.

The captured image 30 that has been captured is displayed on the display 55 by the display control unit 54c. In this case, since the imaging guide 33 is already displayed on the display 55, the captured image 30 and the imaging guide 33 are displayed on the display 55 in a superimposed manner.

The determination processing unit 54d determines whether or not the captured image 30 is suitable for the outer diameter type determination of the endoscope 14 (step S303). Here, in a case in which the mark portion 31 in the captured image 30 protrudes beyond the region of the imaging guide 33, the captured image 30 is not suitable for the outer diameter type determination of the endoscope 14 and cannot be employed. In such a case, the determination processing unit 54d determines that the captured image 30 is not suitable for the outer diameter type determination of the endoscope 14. In a case in which the determination processing unit 54d determines that the captured image 30 that has been captured is not suitable for the outer diameter type determination of the endoscope 14, the user is notified of the fact. Specifically, in a case in which the mark portion 31 in the captured image 30 protrudes beyond the region of the imaging guide 33, the determination processing unit 54d causes the display control unit 54c to perform error display on the display 55. Based on such a notification, the user adjusts the insertion amount of the distal end portion 14a of the endoscope 14 into the adjustment jig 10. After that, the imaging is performed again (step S302). Then, steps S302 and S303 are repeated until the captured image 30 suitable for the outer diameter type determination of the endoscope 14 is obtained. The user may determine whether or not the captured image 30 is suitable for the outer diameter type determination of the endoscope 14, instead of the determination processing unit 54d. In this case, the user repeats steps S302 and S303 until the captured image 30 suitable for the outer diameter type determination of the endoscope 14 is obtained.

Then, in a case in which the captured image 30 suitable for the outer diameter type determination of the endoscope 14 is obtained, that is, in a case in which the captured image 30 in which the mark portion 31 falls within the region of the imaging guide 33 is obtained, the determination processing unit 54d performs the outer diameter type determination of the endoscope 14 (step S304). Specifically, the determination processing unit 54d refers to the correspondence relationship table or the like stored in the storage unit 54e and determines the outer diameter type of the distal end portion 14a of the endoscope 14 based on the shape of the mark portion 31 and the shape of the inner side surface portion 34 in the captured image 30. Then, in a case in which the determination processing unit 54d cannot determine the outer diameter type of the endoscope 14, the process returns from step S305 to step S302, and the chart 18, the mark 17, and the inner side surface 13h of the outer peripheral portion 13 are re-imaged. The case in which the outer diameter type of the endoscope 14 cannot be determined is, for example, a case in which there is no feature amount for comparison corresponding to the feature amount of the shape of the mark portion 31, the shape of the inner side surface portion 34, or the like in the captured image 30 even with reference to the correspondence relationship table or the like.

On the other hand, in step S305, in a case in which the determination processing unit 54d can determine the outer diameter type of the endoscope 14, the process proceeds to step S306, and the determination processing unit 54d causes the display control unit 54c to display the determination result, that is, the value of the outer diameter of the distal end portion 14a, on the display 55 (outputs the value to the outside). Additionally, the determination processing unit 54d causes the image processing unit 54b to create an image signal correction parameter (correction parameter) corresponding to the determined outer diameter type of the endoscope 14 (step S307).

In this manner, the outer diameter of the distal end portion 14a of the endoscope 14 can be determined based on the captured image 30 captured by the endoscope 14. Therefore, it is possible to create an image signal correction parameter corresponding to the type of the endoscope 14. As a result, image signal correction can be performed with greater accuracy.

In addition, by determining whether or not the captured image 30 is suitable for the type determination of the endoscope 14, the type of the endoscope 14 can be determined based on a more suitable captured image 30. As a result, image signal correction can be performed with greater accuracy.

The determination by the determination processing unit 54d can be performed using known image processing techniques, and the determination may be performed using a learning model in machine learning techniques, which has been trained on the position and the size of the mark portion 31 in the captured image 30, or, in a case of necessity, the inner side surface portion 34.

Additionally, similar to the second embodiment, after the outer diameter type determination of the endoscope 14 is performed, the captured image 30 more suitable for creating the image signal correction parameter may be obtained using the imaging guide 33 corresponding to the type of the endoscope 14. That is, the position in the captured image 30 of the imaging guide 33 displayed on the display 55 in a case in which the chart 18 or the like is imaged by the distal end portion 14a of the endoscope 14 may be set based on the oblique angle α of the endoscope 14 obtained from the shape of the mark 17 imaged by the endoscope. Further, similar to the second embodiment, a notification of the imaging state may be issued based on the comparison result between the shape of the mark 17 imaged by the endoscope 14 and the imaging guide 33.

### Ninth Embodiment

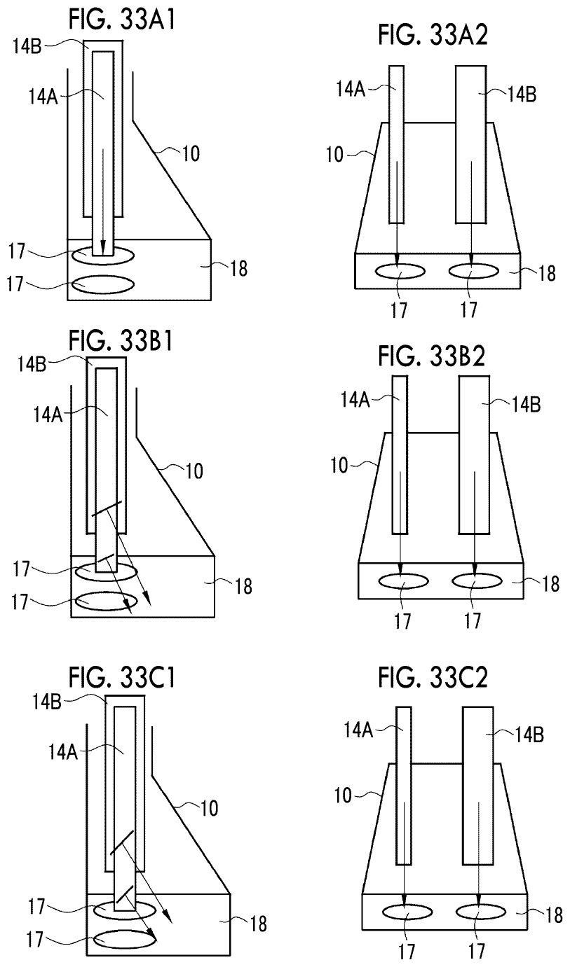

In the eighth embodiment, in the adjustment jig 10, two insertion holes 15a and 15b are provided at substantially the center position between the top surface portion 13a and the bottom surface portion 13b of the outer peripheral portion 13. Meanwhile, in a ninth embodiment, as shown in FIGS. 33A1 to 33C2, the chart 18 is disposed on the perpendicular plane perpendicular to the insertion direction X of the distal end portion 14a. The two insertion holes 15a and 15b are provided at a position close to any one of the top surface portion 13a or the bottom surface portion 13b of the outer peripheral portion 13.

Here, the relationship between the endoscope 14 and the adjustment jig 10 in a case in which the chart 18 and the like are imaged using the endoscope 14 will be described, and since the other parts are the same as those in the first embodiment, the second embodiment, or the eighth embodiment, the description thereof will be omitted.

FIGS. 33A1 to 33C2 are conceptual diagrams of a state in which the distal end portion 14a of the endoscope 14 is inserted into the adjustment jig 10 in order to perform the outer diameter type determination of the endoscope 14. In FIGS. 33A1 to 33C2, two types of endoscopes 14 (for example, an endoscope with an outer diameter of 5 mm and an endoscope with an outer diameter of 10 mm) are depicted as being simultaneously inserted, but in practice, the outer diameter type determination is performed one by one.

FIGS. 33A1, 33B1, and 33C1 are conceptual diagrams of the adjustment jig 10 as viewed from the Z direction (refer to FIG. 3), and FIGS. 33A2, 33B2, and 33C2 are conceptual diagrams of the adjustment jig 10 as viewed from the Y direction (refer to FIG. 3). The endoscopes 14A and 14B in FIGS. 33A1 and 33A2 are direct-view scopes. Additionally, the endoscopes 14A and 14B in FIGS. 33B1 and 33B2 are oblique-view scopes with an oblique angle α=30 degrees. Further, the endoscopes 14A and 14B in FIGS. 33C1 and 33C2 are oblique-view scopes with an oblique angle α=45 degrees.

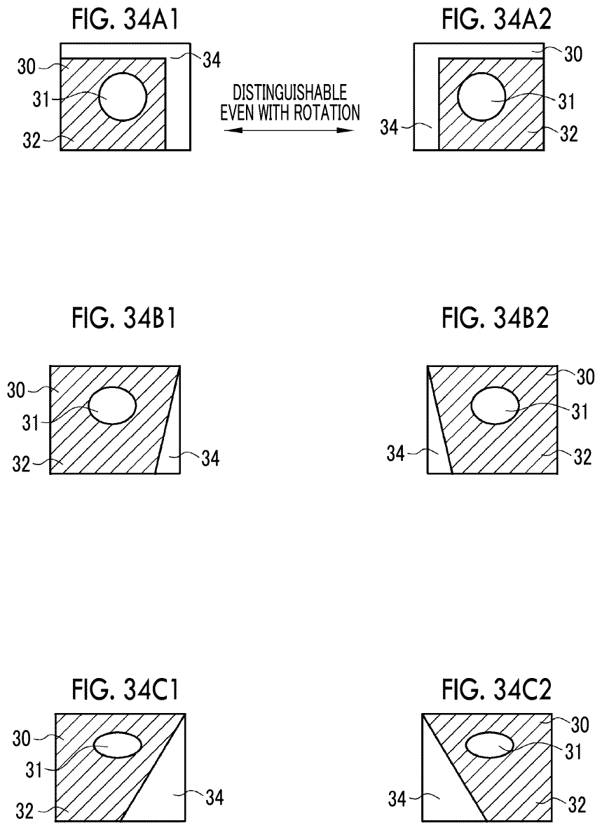

The captured image 30 of the endoscope 14B shown in FIGS. 33A1 and 33A2 is shown in FIG. 34A1. In addition, the captured image 30 of the endoscope 14A shown in FIGS. 33A1 and 33A2 is shown in FIG. 34A2. Further, the captured image 30 of the endoscope 14B shown in FIGS. 33B1 and 33B2 is shown in FIG. 34B1. Additionally, the captured image 30 of the endoscope 14A shown in FIGS. 33B1 and 33B2 is shown in FIG. 34B2. Further, the captured image 30 of the endoscope 14B shown in FIGS. 33C1 and 33C2 is shown in FIG. 34C1. Furthermore, the captured image 30 of the endoscope 14A shown in FIGS. 33C1 and 33C2 is shown in FIG. 34C2.

As is clear from the six captured images 30 shown in FIGS. 34A1 to 34C2, in the captured images 30 for each combination of the oblique angle α and the outer diameter, the shapes of the mark portion 31 and the inner side surface portion 34 of the outer peripheral portion 13 are all different. More specifically, the degree of collapse of the mark portion 31 and the position of the inner side surface portion 34 are different. The shape of the inner side surface portion 34 of the outer peripheral portion 13 is different because the distal end portions 14a of the endoscopes 14 with different outer diameters are inserted into different insertion holes 15a and 15b, resulting in different dispositions in the adjustment jig 10. Therefore, a relationship between the distinction of the outer diameters of the endoscopes 14A and 14B and the oblique angle α, and the shapes of the mark portion 31 and the inner side surface portion 34 of the outer peripheral portion 13 in the captured image 30 is obtained in advance, and the relationship is stored in the storage unit 54e as the correspondence relationship table or the like. Then, by referring to the correspondence relationship table or the like to compare the shapes of the mark portion 31 and the inner side surface portion 34 of the outer peripheral portion 13 in the captured image 30 that has been captured, with the shapes of the mark portion and the inner side surface portion in the correspondence relationship table or the like, the outer diameter type determination of the endoscope 14 inserted into the adjustment jig 10 can be performed. Here, the outer diameter type determination has been described, but it is obvious that the oblique angle determination can also be performed by the same procedure.

In addition, as is clear from the six captured images 30 shown in FIGS. 34A1 to 34C2, the captured images 30 are different even at the same oblique angle. For example, since the insertion holes 15a and 15b of the adjustment jig 10 are provided at a position close to any one of the top surface portion 13a or the bottom surface portion 13b of the outer peripheral portion 13, in a case in which the endoscope 14 is a direct-view scope, the top surface portion 13a or the bottom surface portion 13b of the outer peripheral portion 13 is captured in the captured image 30. Therefore, even with the same direct-view scope, in a case in which the outer diameter is different, the captured image 30 in FIG. 34A1 and the captured image 30 in FIG. 34A2 are different. Further, even with the same oblique angle, the captured image 30 does not match the captured image 30 with the other outer diameter even in a case in which the captured image 30 is rotated. That is, even in a case in which the distal end portion 14a of the endoscope 14 is rotated, the captured image 30 captured by the endoscope 14 and the captured image 30 captured by the endoscope 14 with the other outer diameter do not match.

Here, the captured image 30 in FIG. 31A1 and the captured image 30 in FIG. 31A2 in the eighth embodiment have different left-right orientations, but are the same captured image 30. That is, in a case in which one captured image 30 is rotated by 180 degrees, the captured image 30 matches the other captured image 30. Therefore, in order to accurately perform the outer diameter type determination, it is necessary to align the circumferential orientation of the distal end portion 14a with a predetermined orientation in a case in which the distal end portion 14a of the endoscope 14 is inserted into the insertion hole 15. On the other hand, in the present embodiment, since the captured image 30 in FIG. 34A1 and the captured image 30 in FIG. 34A2 are different, it is possible to perform the outer diameter type determination without aligning the circumferential orientation of the distal end portion 14a with the predetermined orientation in a case in which the distal end portion 14a of the endoscope 14 is inserted into the insertion hole 15. Therefore, it is possible to create an image signal correction parameter corresponding to the type of the endoscope 14. As a result, image signal correction can be performed with greater accuracy.

The shapes of the mark portion 31 and the inner side surface portion 34 of the outer peripheral portion 13 in the captured image 30, which are associated with the distinction of the outer diameters of the endoscopes 14A and 14B and the oblique angle α, may be a plurality of types of shapes obtained by rotating the distal end portions 14a of the endoscopes 14A and 14B in the adjustment jig 10. As a result, the outer diameter type determination can be performed even from the captured image 30 of the distal end portion 14a in various circumferential orientations.

### Tenth Embodiment

In the eighth embodiment and the ninth embodiment, the adjustment jig 10 is used in which the chart 18 is disposed on the perpendicular plane perpendicular to the insertion direction X of the distal end portion 14a of the endoscope 14 (inclination angle β=0 degrees). Meanwhile, in a tenth embodiment, the adjustment jig 10 is used in which the chart 18 is disposed to be inclined with respect to the perpendicular plane perpendicular to the insertion direction X of the distal end portion 14a of the endoscope 14.

Here, the relationship between the endoscope 14 and the adjustment jig 10 in a case in which the chart 18 and the like are imaged using the endoscope 14 will be described, and since the other parts are the same as those in the first embodiment, the second embodiment, or the eighth embodiment, the description thereof will be omitted.

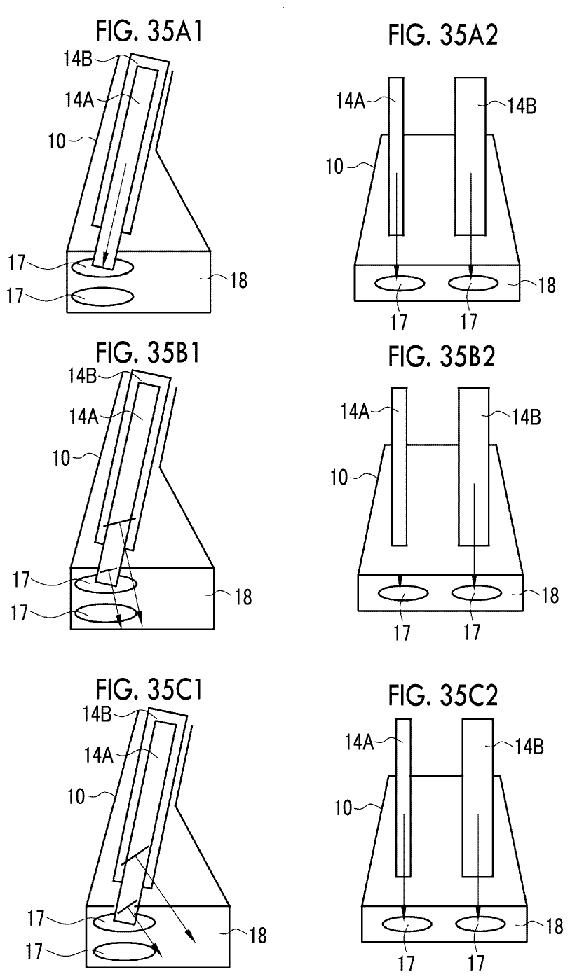

FIGS. 35A1 to 35C2 are conceptual diagrams of a state in which the distal end portion 14a of the endoscope 14 is inserted into the adjustment jig 10 in order to perform the outer diameter type determination of the endoscope 14. In FIGS. 35A1 to 35C2, two types of endoscopes 14 (for example, an endoscope with an outer diameter of 5 mm and an endoscope with an outer diameter of 10 mm) are depicted as being simultaneously inserted, but in practice, the outer diameter type determination is performed one by one.

FIGS. 35A1, 35B1, and 35C1 are conceptual diagrams of the adjustment jig 10 as viewed from the Z direction (refer to FIG. 3), and FIGS. 35A2, 35B2, and 35C2 are conceptual diagrams of the adjustment jig 10 as viewed from the Y direction (refer to FIG. 3). The endoscopes 14A and 14B in FIGS. 35A1 and 35A2 are direct-view scopes. Additionally, the endoscopes 14A and 14B in FIGS. 35B1 and 35B2 are oblique-view scopes with an oblique angle α=30 degrees. Further, the endoscopes 14A and 14B in FIGS. 35C1 and 35C2 are oblique-view scopes with an oblique angle α=45 degrees.

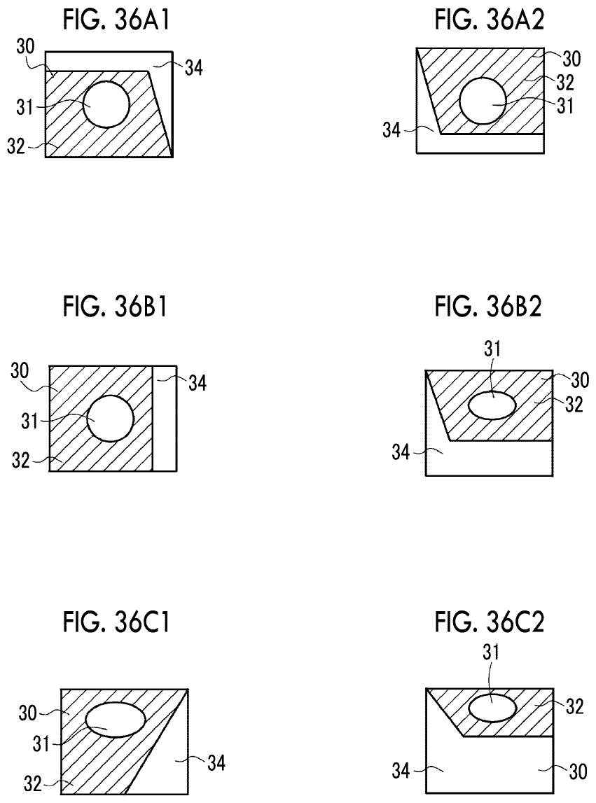

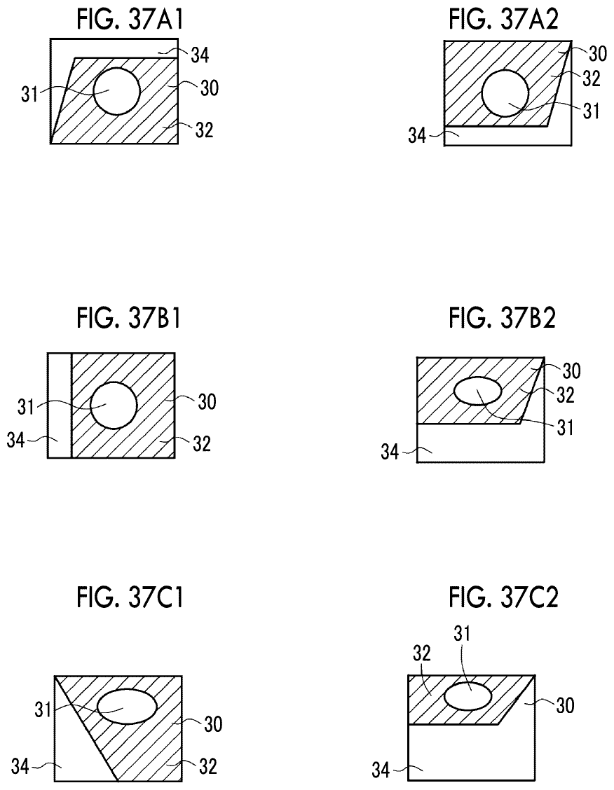

The captured images 30 of the endoscope 14B shown in FIGS. 35A1 and 35A2 are shown in FIGS. 36A1 and 36A2. In addition, the captured images 30 of the endoscope 14A shown in FIGS. 35A1 and 35A2 are shown in FIGS. 37A1 and 37A2. Further, the captured images 30 of the endoscope 14B shown in FIGS. 35B1 and 35B2 are shown in FIGS. 36B1 and 36B2. Additionally, the captured images 30 of the endoscope 14A shown in FIGS. 35B1 and 35B2 are shown in FIGS. 37B1 and 37B2. Further, the captured images 30 of the endoscope 14B shown in FIGS. 35C1 and 35C2 are shown in FIGS. 36C1 and 36C2. Furthermore, the captured images 30 of the endoscope 14A shown in FIGS. 35C1 and 35C2 is shown in FIGS. 37C1 and 37C2. Here, the captured images 30 in FIGS. 36A1, 36B1, and 36C1 and FIGS. 37A1, 37B1, and 37C1 are captured images 30 in a case in which the circumferential orientation of the distal end portion 14a of the endoscope 14 is in the positive direction. On the other hand, the captured images 30 in FIGS. 36A2, 36B2, and 36C2 and FIGS. 37A2, 37B2, and 37C2 are captured images 30 in a case in which the circumferential orientation of the distal end portion 14a of the endoscope 14 is in the reverse direction.

As is clear from the six captured images 30 shown in FIGS. 36A1 to 36C2 and the six captured images 30 shown in FIGS. 37A1 to 37C2, in the captured images 30 for each combination of the oblique angle α, the outer diameter, and the circumferential orientation of the distal end portion 14a, the shapes of the mark portion 31 and the inner side surface portion 34 of the outer peripheral portion 13 are all different. More specifically, the degree of collapse of the mark portion 31 and the position of the inner side surface portion 34 are different. Therefore, a relationship between the distinction of the outer diameter of the endoscope 14 and the oblique angle α, and the shapes of the mark portion 31 and the inner side surface portion 34 of the outer peripheral portion 13 in the captured image 30 is obtained in advance, and the relationship is stored in the storage unit 54e as the correspondence relationship table or the like. Then, by referring to the correspondence relationship table or the like to compare the shapes of the mark portion 31 and the inner side surface portion 34 of the outer peripheral portion 13 in the captured image 30 that has been captured, with the shapes of the mark portion and the inner side surface portion in the correspondence relationship table or the like, the outer diameter type determination of the endoscope 14 inserted into the adjustment jig 10 can be performed. Here, the outer diameter type determination has been described, but it is obvious that the oblique angle determination can also be performed by the same procedure.

In addition, since the adjustment jig 10 is used in which the insertion holes 15a and 15b of the adjustment jig 10 are provided at a position close to any one of the top surface portion 13a or the bottom surface portion 13b of the outer peripheral portion 13, and the chart 18 is disposed to be inclined with respect to the perpendicular plane perpendicular to the insertion direction X of the distal end portion 14a of the endoscope 14, two surfaces of the outer peripheral portion 13, that is, the top surface portion 13a or the bottom surface portion 13b, and the right side surface portion 13c or the left side surface portion 13d, are captured. Therefore, the captured images 30 of FIGS. 36A1 to 36C2 and FIGS. 37A1 to 37C2 are all different. Accordingly, in a case in which the distal end portion 14a of the endoscope 14 is inserted into the insertion hole 15, it is possible to perform the outer diameter type determination without aligning the circumferential orientation of the distal end portion 14a.

### Eleventh Embodiment

In the tenth embodiment, the same mark 17 is imaged by the direct-view scope and the oblique-view scope. On the other hand, in an eleventh embodiment, the mark 17 to be imaged by the direct-view scope and the oblique-view scope is changed. Therefore, the chart 18 of the present embodiment is provided with four marks 17. Specifically, there are two marks 17 captured by each of the direct-view scope and the oblique-view scope, and the two marks 17 are provided on the chart 18 to be parallel to the right side surface portion 13c or the left side surface portion 13d.

Here, the relationship between the endoscope 14 and the adjustment jig 10 in a case in which the chart 18 and the like are imaged using the endoscope 14 will be described, and since the other parts are the same as those in the first embodiment, the second embodiment, or the eighth embodiment, the description thereof will be omitted.

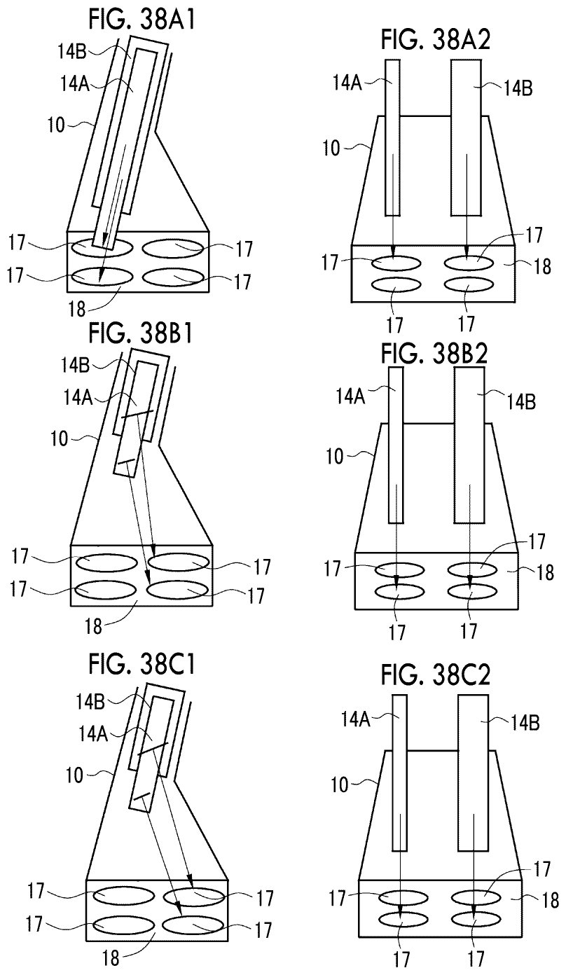

FIGS. 38A1 to 38C2 is a conceptual diagram of a state in which the distal end portion 14a of the endoscope 14 is inserted into the adjustment jig 10 in order to perform the outer diameter type determination of the endoscope 14. In FIGS. 38A1 to 38C2, two types of endoscopes 14 (for example, an endoscope with an outer diameter of 5 mm and an endoscope with an outer diameter of 10 mm) are depicted as being simultaneously inserted, but in practice, the outer diameter type determination is performed one by one.

FIGS. 38A1, 38B1, and 38C1 are conceptual diagrams of the adjustment jig 10 as viewed from the Z direction (refer to FIG. 3), and FIGS. 38A2, 38B2, and 38C2 are conceptual diagrams of the adjustment jig 10 as viewed from the Y direction (refer to FIG. 3). The endoscopes 14A and 14B in FIGS. 38A1 and 38A2 are direct-view scopes. Additionally, the endoscopes 14A and 14B in FIGS. 38B1 and 38B2 are oblique-view scopes with an oblique angle α=30 degrees. Further, the endoscopes 14A and 14B in FIGS. 38C1 and 38C2 are oblique-view scopes with an oblique angle α=45 degrees.

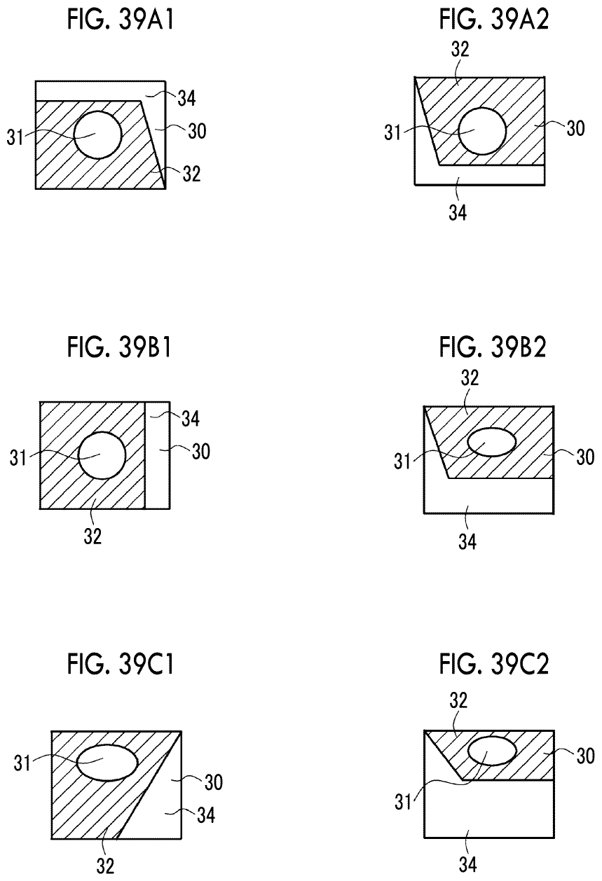

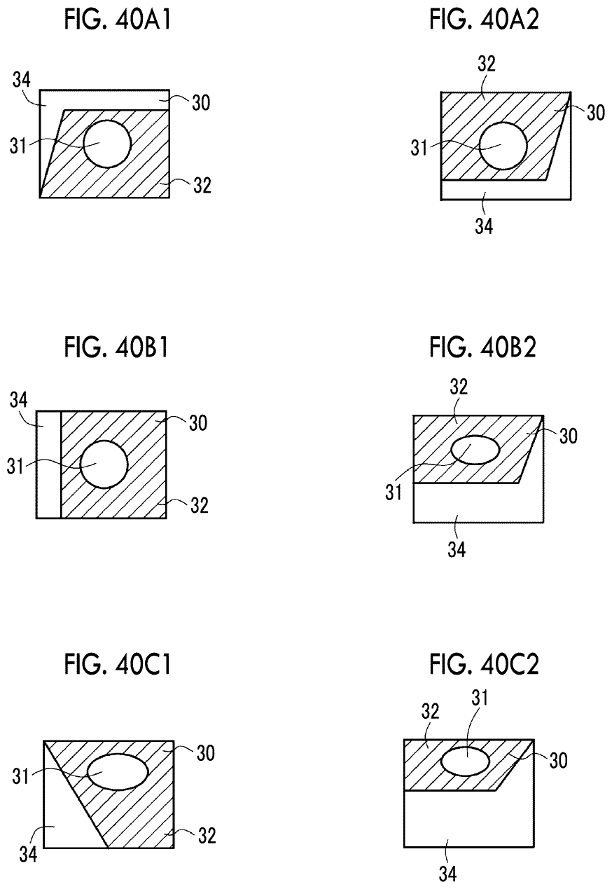

The captured images 30 of the endoscope 14B shown in FIGS. 38A1 and 38A2 are shown in FIGS. 39A1 and 39A2. In addition, the captured images 30 of the endoscope 14A shown in FIGS. 38A1 and 38A2 are shown in FIGS. 40A1 and 40A2. Further, the captured images 30 of the endoscope 14B shown in FIGS. 38B1 and 38B2 are shown in FIGS. 39B1 and 39B2. Additionally, the captured images 30 of the endoscope 14A shown in FIGS. 38B1 and 38B2 are shown in FIGS. 40B1 and 40B2. Further, the captured images 30 of the endoscope 14B shown in FIGS. 38C1 and 38C2 are shown in FIGS. 39C1 and 39C2. Furthermore, the captured images 30 of the endoscope 14A shown in FIGS. 38C1 and 38C2 is shown in FIGS. 40C1 and 40C2. Here, the captured images 30 in FIGS. 39A1, 39B1, and 39C1 and FIGS. 40A1, 40B1, and 40C1 are captured images 30 in a case in which the circumferential orientation of the distal end portion 14a of the endoscope 14 is in the positive direction. On the other hand, the captured images 30 in FIGS. 39A2, 39B2, and 39C2 and FIGS. 40A2, 40B2, and 40C2 are captured images 30 in a case in which the circumferential orientation of the distal end portion 14a of the endoscope 14 is in the reverse direction.

As is clear from the six captured images 30 shown in FIGS. 39A1 to 39C2 and the six captured images 30 shown in FIGS. 40A1 to 40C2, in the captured images 30 for each combination of the oblique angle α, the outer diameter, and the circumferential orientation of the distal end portion 14a, the shapes of the mark portion 31 and the inner side surface portion 34 of the outer peripheral portion 13 in the captured image 30 are all different. More specifically, the degree of collapse of the mark portion 31 and the position of the inner side surface portion 34 are different. Therefore, a relationship between the distinction of the thickness of the endoscope 14 and the oblique angle α, and the shapes of the mark portion 31 and the inner side surface portion 34 of the outer peripheral portion 13 in the captured image 30 is obtained in advance, and the relationship is stored in the storage unit 54e as the correspondence relationship table or the like. Then, by referring to the correspondence relationship table or the like to compare the shapes of the mark portion 31 and the inner side surface portion 34 of the outer peripheral portion 13 in the captured image 30 that has been captured, with the shapes of the mark portion and the inner side surface portion in the correspondence relationship table or the like, the outer diameter type determination of the endoscope 14 inserted into the adjustment jig 10 can be performed. Here, the outer diameter type determination has been described, but it is obvious that the oblique angle determination can also be performed by the same procedure.

In addition, since the adjustment jig 10 is used in which the insertion holes 15a and 15b of the adjustment jig 10 are provided at a position close to any one of the top surface portion 13a or the bottom surface portion 13b of the outer peripheral portion 13, and the chart 18 is disposed to be inclined with respect to the perpendicular plane perpendicular to the insertion direction X of the distal end portion 14a of the endoscope 14, the two surfaces of the outer peripheral portion 13, that is, the top surface portion 13a or the bottom surface portion 13b, and the right side surface portion 13c or the left side surface portion 13d, are captured. Therefore, the images 30 of FIGS. 39A1 to 39C2 and FIGS. 40A1 to 40C2 are all different. Accordingly, in a case in which the distal end portion 14a of the endoscope 14 is inserted into the insertion hole 15, it is possible to perform the outer diameter type determination without aligning the circumferential orientation of the distal end portion 14a.

Additionally, in the above-mentioned embodiments, the value of the oblique angle has been described with specific values, but it is also possible to determine the type even with other angles.

Moreover, in the above-mentioned embodiments, the value of the outer diameter of the distal end portion 14a of the endoscope 14 has been described with specific values, but it is also possible to determine the type even with other outer diameter values.

### Explanation of References

10: adjustment jig
11: insertion portion
11a, 11b: guide portion
12: lid portion
12a: outer surface
12b: inner surface
12c: protruding portion
12e: lid portion base
13: outer peripheral portion
13a: top surface portion
13b: bottom surface portion
13c: right side surface portion
13d: left side surface portion
13e: partition
13f: magnet insertion portion
13g: recessed portion
13h: inner side surface
14: endoscope
14a: distal end portion
14b: distal end surface
14A: small-diameter endoscope
14B: large-diameter endoscope
15: insertion hole
15a: first insertion hole
15b: second insertion hole
16a, 16b: internal space
17: mark
18: chart
30: image
31: mark portion
32: chart portion
33: imaging guide
34: inner side surface portion
50: endoscope system
52: camera head
53: light source device
54: processor device
54a: central control unit
54b: image processing unit
54c: display control unit
54d: determination processing unit
54e: storage unit
55: display
56: input device
57: connector for light source device
58: connector for camera head
d1, d2: outer diameter
α: oblique angle
β: inclination angle
m: optical axis direction
n: axial direction of distal end portion

What is claimed is:

1. A processor device that images a chart including a mark provided in an endoscope color adjustment jig, and determines an oblique angle of an endoscope by a processor, the processor being configured to:
based on a shape of the mark imaged by the endoscope inserted into the endoscope color adjustment jig, determine the oblique angle of the endoscope.

2. The processor device according to claim 1,
wherein the endoscope color adjustment jig includes an insertion portion that includes at least one insertion hole into which a distal end portion of the endoscope is inserted, a lid portion that is disposed to face the insertion portion in an insertion direction of the distal end portion, and an outer peripheral portion that is in contact with the insertion portion and the lid portion to form an internal space with the insertion portion and the lid portion,
the lid portion includes the chart on an inner surface facing the internal space, and
the processor is configured to, based on a shape of an inner side surface of the outer peripheral portion imaged by the endoscope, determine the oblique angle of the endoscope.

3. The processor device according to claim 2,
wherein the processor is configured to display, on a display, an imaging guide for adjusting a distance between the endoscope and the mark in a case in which the mark is imaged by the endoscope inserted from the insertion portion.

4. The processor device according to claim 3,
wherein the processor is configured to issue a notification of an imaging state based on a comparison result between the shape of the mark imaged by the endoscope and the imaging guide.

5. The processor device according to claim 1,
wherein the processor is configured to, based on the shape of the mark in at least one frame of a video obtained by imaging the mark with the endoscope, determine the oblique angle of the endoscope.

6. The processor device according to claim 1,
wherein the processor is configured to, based on a still image obtained by imaging the mark with the endoscope in response to a still image acquisition instruction, determine the oblique angle of the endoscope.

7. The processor device according to claim 1,
wherein the processor is configured to, based on a comparison result between a feature amount of the shape of the mark imaged by the endoscope and a feature amount for comparison, determine the oblique angle of the endoscope.

8. The processor device according to claim 1,
wherein the processor is configured to use a learning model trained on the shape of the mark through machine learning to, based on the shape of the mark imaged by the endoscope, determine the oblique angle of the endoscope.

9. The processor device according to claim 1,
wherein the processor is configured to output a result of the determination of the oblique angle of the endoscope to an outside.

10. The processor device according to claim 1,
wherein the processor is configured to decide on a correction parameter for correcting a captured image signal obtained by imaging the mark with the endoscope, based on a result of the determination of the oblique angle of the endoscope.

11. The processor device according to claim 3,
wherein the processor is configured to set a position of the imaging guide based on a result of the determination of the oblique angle of the endoscope.

12. The processor device according to claim 11,
wherein the processor is configured to issue a notification of an imaging state based on a comparison result between the shape of the mark imaged by the endoscope and the imaging guide.

13. The processor device according to claim 1,
wherein the endoscope color adjustment jig is disposed such that the chart is inclined with respect to a perpendicular plane perpendicular to an insertion direction of a distal end portion of the endoscope.

14. The processor device according to claim 2,
wherein the endoscope color adjustment jig is disposed such that the chart is inclined with respect to a perpendicular plane perpendicular to the insertion direction of the distal end portion of the endoscope, and an inclination angle β of the endoscope color adjustment jig is set in advance such that, in a case in which the distal end portion of the endoscope inserted into the insertion portion is rotated in a circumferential direction, a difference appears in the shape of the mark imaged by endoscopes with different oblique angles α.

15. The processor device according to claim 2,
wherein the endoscope color adjustment jig is disposed such that the chart is inclined with respect to a perpendicular plane perpendicular to the insertion direction of the distal end portion of the endoscope, and a position of the mark on the chart is set in advance such that, in a case in which the distal end portion of the endoscope inserted into the insertion portion is rotated in a circumferential direction, a difference appears in the shape of the mark imaged by endoscopes with different oblique angles α.

16. The processor device according to claim 2,
wherein the processor is configured to issue a notification that a circumferential position of the distal end portion of the endoscope should be positioned as the distal end portion is inserted into the insertion portion, before determining the oblique angle.

17. The processor device according to claim 1,
wherein the chart of the endoscope color adjustment jig includes a plurality of the marks.

18. A processor device that performs imaging using an endoscope color adjustment jig, and determines an outer diameter type of an endoscope by a processor, the endoscope color adjustment jig including:
insertion portions that include at least two types of insertion holes with different diameters, into which distal end portions of endoscopes with different outer diameters are inserted;
a lid portion that is disposed to face the insertion portions in an insertion direction of the distal end portion; and
an outer peripheral portion that is in contact with the insertion portions and the lid portion to form an internal space with the insertion portions and the lid portion,
the lid portion including a chart that includes at least two marks facing the respective insertion portions on an inner surface facing the internal space,
the marks being located at positions set in advance on the chart such that differences appear in a shape of the mark and a shape of an inner side surface of the outer peripheral portion, which are imaged by endoscopes of different outer diameter types that have been inserted into the insertion portions,
the processor being configured to:
based on the shape of the mark and the shape of the inner side surface of the outer peripheral portion, which are imaged by the endoscope inserted from the insertion portion, determine the outer diameter type of the endoscope.
